(19) United States
(12) Patent Application Publication
(10) Pub. No.: US 2023/0159906 A1
(43) Pub. Date: May 25, 2023

(54) Protease-controlled Secretion and Display of Intercellular Signals

(71) Applicant: The Board of Trustees of the Leland Stanford Junior University, Stanford, CA (US)

(72) Inventors: Alexander Elias Vlahos, Los Altos, CA (US); Xiaojing Gao, Redwood City, CA (US); Jeewoo Kang, Redwood City, AA (US)

(21) Appl. No.: 17/985,624

(22) Filed: Nov. 11, 2022

Related U.S. Application Data
(60) Provisional application No. 63/282,689, filed on Nov. 24, 2021.

(51) Int. Cl. C12N 9/50; C07K 14/705; C07K 14/72

(52) U.S. Cl. CPC C12N 9/506; C12Y 304/22044; C07K 14/70517; C07K 14/723; C07K 2319/04; C07K 2319/50; C07K 2319/036; A61K 48/00

(57) ABSTRACT

To program intercellular communication for biomedicine, it is crucial to regulate the secretion and surface display of signaling proteins. If such regulations are at the protein level, there are additional advantages, including compact delivery and direct interactions with endogenous signalling pathways. A modular, generalizable design is provided called Retained Endoplasmic Cleavable Secretion (RELEASE), with engineered proteins retained in the endoplasmic reticulum and displayed/secreted in response to specific proteases. The design allows functional regulation of multiple synthetic and natural proteins by synthetic protease circuits to realize diverse signal processing capabilities, including logic operation and threshold tuning. By linking RELEASE to additional novel sensing and processing circuits, one would be able to achieve elevated protein secretion in response to “undruggable” oncogene KRAS mutants. RELEASE enables the local, programmable delivery of intercellular cues for a broad variety of fields such as neurobiology, cancer immunotherapy and cell transplantation.

### CROSS-REFERENCE TO RELATED APPLICATIONS

This application claims priority from U.S. Provisional Patent Application 63/282,689 filed Nov. 24, 2021, which is incorporated herein by reference.

### STATEMENT OF GOVERNMENT SPONSORED SUPPORT

This invention was made with Government support under contract EB027723 awarded by the National Institutes of Health. The Government has certain rights in the invention.

### FIELD OF THE INVENTION

This invention relates to compositions and methods of protease-controlled secretion and display of intercellular signals.

### BACKGROUND OF THE INVENTION

Synthetic biology aspires to create biomolecular circuits that can sense the state of cells, process the information, and then deliver therapeutic outputs accordingly. This vision has been enhanced by the creation of protein-based circuits. Protein-based circuits have advantages such as fast operation, compact delivery, and robust, context-independent performance compared to traditional transcriptional circuits. However, these protein circuits have operated in the cytosol, and there remains an urgent need for a design that enables protein-level control of intercellular communication, often required at the “respond” step in “sense-process-respond”.

Cell-cell communication is essential for diverse biological processes, such as the generation of immunological responses, cell differentiation and tissue development, the maintenance of physiological homeostasis, and cancer development. Intercellular communication is typically implemented by secreted molecules, including hormones and cytokines.

To take cancer immunotherapy as an example, an ideal application would be to introduce a protein circuit that can sense the cancerous state of a cell, secrete immunostimulatory signals with temporal and quantitative precision to mobilize the immune system while lysing the cell, and therefore turn these cells into vaccines against other similarly cancerous cells. This would not only avoid the toxic effects associated with the systemic delivery of immunomodulating proteins, but also match the complex, dynamic immune process one would be trying to control. In contrast, of the current local delivery methods, neither nanoparticle, or biomaterial-based delivery platforms can fulfill the aforementioned functions that circuits can deliver.

The present invention advances technology with new protein circuits that addresses the need in the art.

### SUMMARY OF THE INVENTION

The present invention provides a generalized protease-responsive platform called RELEASE to control the secretion and display of proteins. RELEASE is compatible with protein-level circuit operations, and enables plug-and-play control of various outputs using a variety of inputs. For all these examples, the inventors switched the input and output (RELEASE) components, while keeping the intermediate protease chassis intact—without any re-optimization. This highlights the modularity of using protease-based sensors, protease circuits, and RELEASE to engineer sense-and-response capabilities.

When adapting RELEASE for new applications, all one needs is a protein-mediated dimerization event that could be harnessed to reconstitute protease activity. One could therefore tap into additional synthetic receptors platforms that rely on ligand-induced dimerization, such the Generalized Extracellular Molecule Sensor (GEMS), or Tango. This invention demonstrates that one can use intermediate proteases to propagate protease signal from the cell membrane to the ER to activate RELEASE, suggesting that using alternative motifs may allow for signal propagation from other subcellular locations, such as nucleus or mitochondria, to ER. Because the components of the conventional protein secretion pathway are conserved among different cell types and species, RELEASE functions in these different contexts as well.

RELEASE enables novel therapeutic modalities. For example, the KRAS-sensing circuit can be used to selectively express immunostimulatory signals (such as IL-12, surface T-cell engagers, and anti-PD1) to mark cancer cells for T-cell mediated destruction without affecting normal cells. The selectivity of the circuit can be further improved using additional proteases through quantitative thresholding or logic operations. For the latter, many RAS-driven cancers harbour additional mutations to tumor suppressor proteins, such as p53. One could use split proteases fused to nanobodies that have preferential binding to mutant p53, to activate RELEASE only when both mutant KRAS and mutant p53 are simultaneously present, via AND logic. An additional benefit is that the protease circuit components can be encoded within single mRNA transcripts that do not pose the risk of insertional mutagenesis.

RELEASE will also expedite other potential therapeutic applications in fields as diverse as neurobiology, developmental biology, immunology, tissue engineering, and transplantation, to name a few. To take a third and last example, in addition to the cancer immunotherapy and neuronal silencing applications discussed above, RELEASE can be used to create sense-and-respond cells to control immunomodulating cytokines and growth factors important for graft acceptance, such as IL-10 and TGF-β, which cannot normally be delivered systemically due to their pleiotropic and off-target effects. Co-delivering these engineered cells with therapeutic cells, such as pancreatic islets may be a suitable approach create engineered tissue implants that can engraft without the need for systemic immunosuppression. The herein provided plug-and-play sense and secretion components using RELEASE would allow for the programming of such communications with unprecedented specificity and precision.

In one embodiment, a composition is provided for protease-controlled secretion of intercellular signals of a protein of interest. The composition has:
A transmembrane anchor domain capable of being inserted to or retained by an Endoplasmic Reticulum (ER) membrane. The ER membrane distinguishes an inside to the ER membrane and an outside to the ER membrane.
A luminal facing linker containing a furin endoprotease cut site. The luminal facing linker is capable of making a physical connection with the protein of interest. The furin endoprotease cut site is linked to the transmembrane anchor domain. Once the transmembrane anchor domain is inserted to or retained by the ER membrane the luminal facing linker and the furin endoprotease cut site are located at the inside of the ER membrane.
A cytosolic linker containing a protease cleavage site. Once the transmembrane anchor domain is inserted to or retained by the ER membrane the cytosolic linker and the protease cleavage site are located at the outside of the ER membrane.
   An Endoplasmic Reticulum (ER) retention motif linked to the protease cleavage site of the cytosolic linker.

At the cytosolic linker, the ER retention motif ensures that the protein of interest is actively transported back to the inside of the ER membrane, unless the ER retention motif is removed by a protease. On the luminal facing linker, the protein of interest is initially tethered to the ER membrane through the luminal facing linker and thus coupled to the cytosolic linker and the ER retention motif. The protein of interest tethered to the ER membrane is processed into a soluble form through cleavage by furin in a trans-Golgi apparatus, and secreted.

In another embodiment, a composition is provided for surface expression of intercellular signals of a protein of interest. The composition has:
A transmembrane anchor domain capable of being inserted to or retained by an Endoplasmic Reticulum (ER) membrane. The ER membrane distinguishes an inside to the ER membrane and an outside to the ER membrane.
A luminal facing linker capable of making a physical connection with the protein of interest. The luminal facing linker is linked to the transmembrane anchor domain. Once the transmembrane anchor domain is inserted to or retained by the ER membrane the luminal facing linker is located at the inside of the ER membrane.
A cytosolic linker containing a protease cleavage site. Once the transmembrane anchor domain is inserted to or retained by the ER membrane the cytosolic linker and the protease cleavage site are located at the outside of the ER membrane.
An Endoplasmic Reticulum (ER) retention motif linked to the protease cleavage site of the cytosolic linker.
At the cytosolic linker, the ER retention motif ensures that the protein of interest is actively transported back to the inside of the ER membrane, unless the ER retention motif is removed by a protease. On the luminal facing linker, the protein of interest is initially tethered to the ER membrane and thus coupled to the cytosolic linker and the ER retention motif. The protein of interest tethered to the ER membrane is transported through a conventional secretory pathway, and expressed on the surface of the ER membrane.

In still another embodiment, an immunotherapy method is provided using protease-controlled secretion of intercellular signals of a protein of interest. The method involves inserting or binding a protease-controlling secretion composition to an Endoplasmic Reticulum (ER) membrane so that the protease-controlling secretion composition is retained by the ER membrane. The ER membrane distinguishes an inside to the ER membrane and an outside to the ER membrane. The protease-controlling secretion composition is defined as the composition for protease-controlled secretion of intercellular signals of a protein of interest, as described infra. In this embodiment, the protein of interest is excreted/secreted and can diffuse into a local microenvironment.

In yet another embodiment, an immunotherapy method is provided using protease-controlled surface expression of intercellular signals of a protein of interest. The method involves inserting or binding a protease-controlling surface expression composition to an Endoplasmic Reticulum (ER) membrane so that the protease-controlling secretion composition is retained by the ER membrane. The ER membrane distinguishes an inside to the ER membrane and an outside to the ER membrane. The protease-controlling secretion is defined as the composition for surface expression of intercellular signals of a protein of interest, as described infra. In the embodiment, the protein of interest is excreted/secreted, but still bound to the ER membrane of the cell (typically known as surface expression).

### Definitions

A transmembrane anchor domain is defined as a membrane protein that spans the entire cell membrane. RELEASE should be compatible with any transmembrane anchor domain including, but not limited to the following:
   The first transmembrane anchor domain of the beta-2 adrenergic receptor.
   The transmembrane anchor domain of E-Cadherin.
   The transmembrane anchor domain of P-Cadherin.
   The transmembrane anchor domain of CD4.
   The transmembrane anchor domain of CD8.
   The first transmembrane anchor domain of insulin growth factor receptor 1.
   The transmembrane anchor domain of CD28.
   The transmembrane anchor domain of CD79B.
   The transmembrane anchor domain of ORT2.
   The transmembrane anchor domain of GpA.
   Combinations of 3 transmembrane anchor domains having the first two transmembrane anchor domains of beta-2 adrenergic receptor and the transmembrane anchor domain of CD8. See FIG. 1E for using a single transmembrane anchor domain versus 3 transmembrane anchor domains (tri-transmembrane).
A luminal facing linker is defined as a protein that is primarily comprised of stretches of glycine and serine amino acid residues. The inventors have used flexible linkers spanning 5 amino acids up to 45 amino acids. To avoid repetitive regions some of the longer linkers also include charged residues (aspartic or glutamic acid) to ensure the linker remains soluble.
A furin endoprotease cut site is defined as the target sequence of the furin endoprotease, which is R—X—K/R—R (where X can be any amino acid; R=arginine amino acid, K=lysine amino acid). Additional amino acid residues flanking the cut site can affect the cleavage efficiency of furin.
A cytosolic linker is defined as a protein that is primarily comprised of stretches of glycine and serine amino acid residues. The inventors have used flexible linkers spanning 5 amino acids up to 45 amino acids. To avoid repetitive regions some of the longer linkers also include charged residues (aspartic or glutamic acid) to ensure the linker remains soluble.
A protease cleavage site is defined as the target sequence of the cognate protease.
   For example, to create a HCVP-inducible RELEASE constructs the protease cleave site will be -E-D-V—V—C—C—S-M-S—.
   TEVP-inducible RELEASE constructs will have protease cleavage sites with the following sequence: -E-N-L-Y—F-Q-S—.
   TVMVP-inducible RELEASE constructs will have protease cleavage sites with the following sequence: -E-T-V-R—F-Q-S.
   Examples of data referencing these different protease cleavage sites can be found in FIGS. 1A-H, FIGS. 2A-F FIGS. 3A-F. It should be noted that any protease/protease cleavage sites should be compatible with the RELEASE design.
An Endoplasmic Reticulum (ER) retention motif is defined as a sequence of amino acids that signals the protein to be retained in the ER. The ER retention domains must be facing the cytoplasmic side of the ER membrane. The following three sequences are compatible with RELEASE:
   —K—K—X—X—COOH, where X can be any amino acid and the —COOH denotes that it must be at the C-terminal of RELEASE.
   —R—X—R—, where X can be any amino acid and does not have to be present at the C-terminal of RELEASE.
   The first 29 amino acids of the cytochrome p450 2C1 protein (NH2-MDPVVVLGLCLSCLLLLSLWKQSYGGGKL-), where NH2 denotes that it must be at the N-terminal of RELEASE.
The inventors have used all three ER retention motifs for the purposes of this invention.
A protein of interest is defined as any protein that can be tethered to the luminal facing linker and is context-specific depending on the application.
   For example cytokines: IL-12, IL-2, IL-6, IL-15, TNF-α, IL-10.
      For example reporter proteins: secreted alkaline embryonic phosphatase (SEAP), secretory N-Luciferase, secreted green fluorescent protein (GFP), mCherry.
Immunotherapy is defined by activating the host's immune system to target the cancer cells.
   CAR-T cell therapy can be improved by controlling the local concentration of pro-inflammatory cytokines (e.g. IL-2, IL-12, IL-15) using RELEASE.
   Using RELEASE and synthetic protein sensors to interrogate the cancerous state of a cell, and conditionally lyse oncogenic cells, while programming cytokine secretion to activate a broader local immune response.
   mRNA delivery of RELEASE and synthetic protein circuit to improve cancer vaccines against specific cancer antigens.

### BRIEF DESCRIPTION OF THE DRAWINGS

FIGS. 1A-H show according to an exemplary embodiment of the invention the design of Retained Endoplasmic Retained Secretion (RELEASE). FIG. 1A: Proteins of interest are fused to RELEASE and retained in the ER via the dilysine ER retention domain (diamond). Upon activation or expression of a protease such as TEVP (partial circle O), the ER retention domain is removed (middle panel) and the protein of interest is transported through the constitutive secretory pathway. When reaching the Trans-Golgi Apparatus (right panel), the native furin endoprotease cleaves the linker region allowing the membrane-bound protein to be secreted. FIG. 1B: RELEASE is a modular platform and can be modified to respond to different proteases and regulate different proteins of interest. FIG. 1C: The C-terminal dilysine motif of RELEASE is required for SEAP retention and mutation of the two lysine residues to alanines (KKXX—COOH→AAXX—COOH) increased SEAP secretion. There was no significant difference in signal between RELEASE and control cells without SEAP. FIG. 1D: Co-expression of proteases such as TEVP (partial circle O), or HCVP (partial circle Y) with the respective RELEASE constructs increased SEAP secretion. FIG. 1E: Single transmembrane and tri-transmembrane RELEASE constructs had different cleavage efficiencies to TEVP cleavage. FIG. 1F: The cleavage efficiencies of HCVP RELEASE constructs were also affected by transmembrane selection and was improved by modifying the residues flanking the HCVP cut site with native linker proteins. Based on the steady-state solution of a kinetic model for proteolytic cleavage, the inventors determined that the relation between RELEASE output and the amount of protease plasmids fits the Michaelis-Menten equation. The inventors therefore fit the titration curves using Michaelis-Menten equations and used Kₘ to represent the apparent cleavage efficiency of each design by its corresponding protease. A complete list of the calculated cleavage efficiencies for the different RELEASE constructs can be found in Supplementary Table 1 (For Supplementary Table 1, the reader is referred to priority document U.S. 63/282,689 filed Nov. 24, 2021, which is incorporated herein by reference). FIG. 1G: By removing the furin cut site, RELEASE was amenable to control the surface display of proteins. FIG. 1H: Increased surface display of membrane-bound GFP fused to RELEASE in response to TEVP (left panel) or HCVP (right panel). Each dot represents a biological replicate. Mean values were calculated from four (FIGS. 1C-F) or three replicates (FIG. 1H). The error bars represent+/−SEM. The results are representative of at least two independent experiments; significance was tested using an unpaired two-tailed Student's t-test between the two indicated conditions for each experiment. For experiments with multiple conditions, a one-way ANOVA with a Tukey's post-hoc comparison test was used to assess significance. ***=p<0.001, ****=p<0.0001.

FIGS. 2A-F show according to an exemplary embodiment of the invention RELEASE in circuits. FIG. 2A: Orthogonal operation of RELEASE constructs. FIG. 2B: HEK293 cells were co-transfected with SEAP fused to RELEASE (responsive to TEVP) and GFP fused to RELEASE (responsive to HCVP). SEAP and GFP levels increase in the supernatant when the cognate protease was expressed. FIG. 2C: Tandem insertion of two protease cut sites (top panel) created a RELEASE construct that implemented OR gate logic. If either of the respective proteases were expressed, the dilysine ER retention motif would be removed, and SEAP would be secreted. FIG. 2D: Implementation of AND logic by adding the N-terminal p450 signal anchor sequence as a second ER retention domain, so that both proteases would have to be present to remove both retention domains and allow SEAP to be secreted. FIG. 2E: A two-protease cascade was created where TEVP was required to activate TVMVP, which subsequently cleaved SEAP RELEASE. SEAP secretion increased when TEVP was expressed (right panel). Each dot represents an individual biological replicate. Mean values were calculated from three (FIG. 2B) or four replicates (FIGS. 2C-E). Error bars represent+/−SEM. The results are representative of at least two independent experiments; significance was tested by one-way ANOVA with a Tukey's post-hoc comparison test among the multiple conditions. *=p<0.05, ***=p<0.001, ****=p<0.0001.

FIGS. 3A-F show according to an exemplary embodiment of the invention controlling bioactive proteins using RELEASE. FIG. 3A: The cytokine IL-12 was fused to RELEASE and placed under the control of TVMVP. FIG. 3B: TVMVP significantly increase IL-12 secretion. FIG. 3A: The inwardly rectifying potassium channel Kir2.1 was fused to RELEASE. In addition, the genetically encoded voltage indicator ASAP3 was co-transfected. FIG. 3D: Co-expression of Kir2.1-RELEASE with TEVP resulted in a significant increase in the amount of Kir2.1 expressed on the surface, which was quantified using surface staining for HA and flow cytometry. The surface display of functional Kir2.1 in response to TVMVP was shown to cause hyperpolarization of transfected cells. This was validated by measuring change in the fluorescence intensity of the genetic reporter FIG. 3E: ASAP3, or the chemical dye, FIG. 3F: DiSBAC2(3). Each dot represents an individual biological replicate. Mean values were calculated from four (FIG. 3B) or three replicates (FIGS. 3D-F). Error bars represent+/−SEM. The results are representative of at least two independent experiments. Significance was tested using an unpaired two-tailed Student's t-test between the two indicated conditions for each experiment. **=p<0.01, ***=p<0.001, ****=p<0.0001.

FIGS. 4A-E show according to an exemplary embodiment of the invention RAS-sensing circuit and protease replaying pathways to activate RELEASE. FIG. 4A: To sense active RAS, split TEVP was fused to the RBD domain of c-RAF. RBD-split TEVP binds to active RAS at the membrane surface of the cell where the two protease halves reassociated and reconstituted protease activity. Protease activation is propagated through an intermediate protease to relay the information from the cell membrane to the ER. FIG. 4B: Using protein localization motifs, three different topologies of intermediate protease components were created. Topology 1 uses two caged intermediate TVMVP protease halves in the cytosol. Topology 2 uses the same caged intermediate TVMVP, but with one half of the active protease localized to the membrane. Finally, Topology 3 has one half of the intermediate protease associated with the membrane, and the other half uncaged and present at the ER membrane via the p450 signal anchor sequence. The CC domain present on the uncaged TVMVP half (that was associated with the membrane) drives association with the complementary TVMVP half at the ER. FIG. 4C: There was a significant difference in the amount of SEAP secreted when using intermediate protease topology 3, with and without mutant HRAS-G12V, compared to topologies 1, and 2. FIG. 4D: Schematic of the signal processing of the complete KRAS-sensing circuit. The complete RAS-sensing circuit was activated by RBD-split TEVP interacting with active KRAS-G12V (1). The reconstituted TEV (2) then uncaged the membrane associated split TVMVP, releasing it from the membrane (3). The uncaged TVMVP contains a CC domain, which drives its association with the complementary CC domain present on the other split TVMVP half anchored to ER membrane (4). Finally, the reconstituted TVMVP cleaves the ER retention motif of RELEASE to secrete SEAP (5). FIG. 4E: Using the complete RAS-sensing circuit, we observed a significant increase in SEAP secretion when expressing an active mutant variant KRAS-G12V relative to baseline levels, or wildtype KRAS. This difference was not observed when using an RBD-Split TEVP containing the R89L mutation that reduced the association with active KRAS. Each dot represents an individual biological replicate. Mean values were calculated from four replicates (FIG. 4C, FIG. 4E). The error bars represent+/−SEM. The results are and representative of at least two independent experiments. Significance was tested using an unpaired two-tailed Student's t-test between the two indicated conditions for each experiment. **=p<0.01, ***=p<0.001, ****=p<0.0001.

FIGS. 5A-G show according to an exemplary embodiment of the invention plug-and-play capabilities of RELEASE. FIG. 5A: Any multimerization event, such as ligand-induced receptor dimerization (i.e. MESA receptors, or Tango), protein association, nanobody-bridged dimerization, or light-induced dimerization can be harnessed to reconstitute and activate split proteases. This information can then be processed using CHOMP circuits or even RELEASE itself to produce complex responses. Each component of the engineered can be optimized independently of each other and are not necessarily dependent on the input or output components. To highlight the plug-and-play capabilities of RELEASE, the inventors tested different input and output combinations, while keeping the intermediate CHOMP circuit intact. FIG. 5B: Using the rapalog MESA receptor as the input, SEAP secretion was controlled. IL-12 secretion was induced by FIG. 5C KRAS expression or induction with FIG. 5D rapalog. FIG. 5E: The inventors also observed Kir2.1-mediated hyperpolarization after induction with Rapalog. FIG. 5F: Schematic of CHOMP circuit containing reciprocal inhibition of TVMVP and HCVP to reduce background activity of RELEASE. When the amount of input is low, the ER-associated split TVMVP protease is repressed by the ER-associated HCVP through removing the complementary CC motif, reducing the association with the other split functional half. When the amount of input is high, fully reconstituted TVMVP will be present at higher levels and repress HCVP by removing the core HCVP from its activity-enhancing co-peptide (small yellow pie space). FIG. 5G: Addition of the tuner protease increased the dynamic range of the RAS-sensing circuit, by reducing baseline secretion. Each dot represents an individual biological replicate. Mean values were calculated from four biological replicates (FIGS. 5B-E, FIG. 5G). Error bars represent+/−SEM. The results are representative of at least two independent experiments. Significance was tested using an unpaired two-tailed Student's t-test between the two indicated conditions for each experiment. **=p<0.01, ****=p<0.0001.

FIG. 6 show according to an exemplary embodiment of the invention compatibility of RELEASE with different transmembrane anchor domains. The effects of different transmembrane domains on the retention and secretory function of RELEASE was experimentally validated using the SEAP reporter assay. Each RELEASE variant showed a significant increase in SEAP secretion when co-expressed with TEVP (tall bars always on the right on pair of two bars), relative to when TEVP was absent (short, almost zero height, bars always on the left on pair of two bars). Mean values were calculated from four replicates. The error bars represent+/−SEM. The results are representative of at least two independent experiments; significance was tested using a one-way ANOVA with a Tukey's post-hoc comparison test. ***p<0.001, ****p<0.0001.

### DETAILED DESCRIPTION

Given the importance of intercellular communication, the inventors sought to interface protein circuits with the secretion and display of protein signals. Specifically, because protease activity has emerged as a “common currency” of protein circuits that responds to synthetic and endogenous inputs, it will be ideal to directly control protein secretion using proteases. To design a modular protease-regulated protein secretion system, the inventors adapted aspects of the natural secretion process.

Secreted proteins are typically transported into the Endoplasmic Reticulum (ER), processed in the Golgi apparatus, and finally secreted at the plasma membrane. Some proteins contain signaling motifs (e.g. KDEL for soluble proteins and the cytosol-facing dilysine (—KKXX) or —RXR motifs for membrane proteins) recognized in the early Golgi apparatus, causing the protein to be retrieved, transported retrogradely, and retained in the ER. Other ER-resident proteins, such as cytochrome p450 are retained in at the ER via their signal-anchor transfer sequence. These retention motifs function in their endogenous contexts as well as when fused to normally secreted proteins.

To place ER retention under protease control, the inventors engineered a modular Retained Endoplasmic Cleavable Secretion (RELEASE) platform, compatible with both protein secretion and the surface display of membrane proteins. The inventors validated and engineered the core mechanism of RELEASE, created input-processing capabilities, and then used RELEASE to control physiological outputs. Finally, the inventors combined RELEASE with novel sensing and processing components to respond to internal cell states and external signals via engineered receptors. This invention demonstrates a protein-level control module to directly regulate protein secretion that is compatible with pre-existing protein components to program therapeutic circuits for cancer immunotherapy and transplantation in the future.

Results

Engineering RELEASE for Protein Secretion and Expression

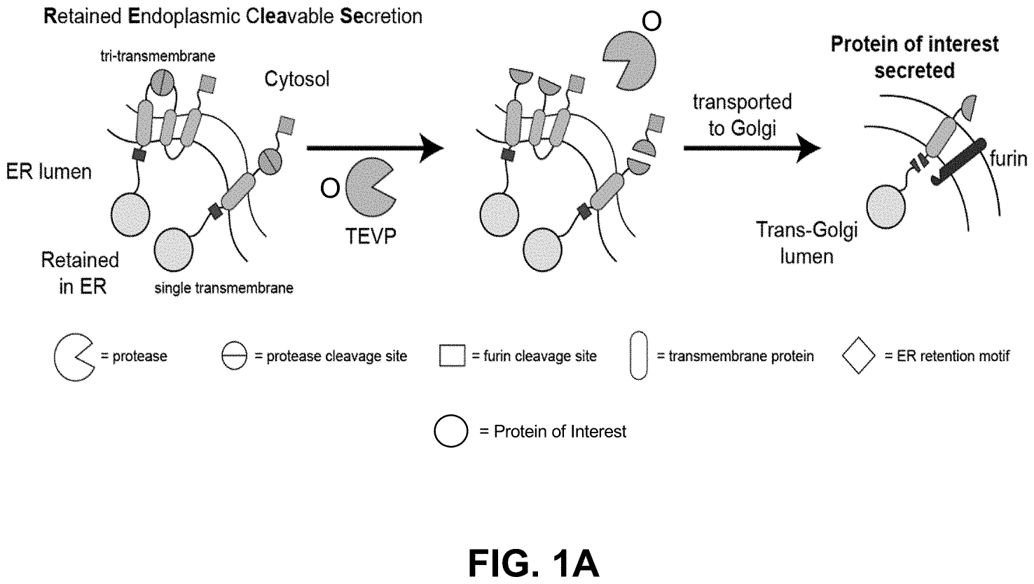

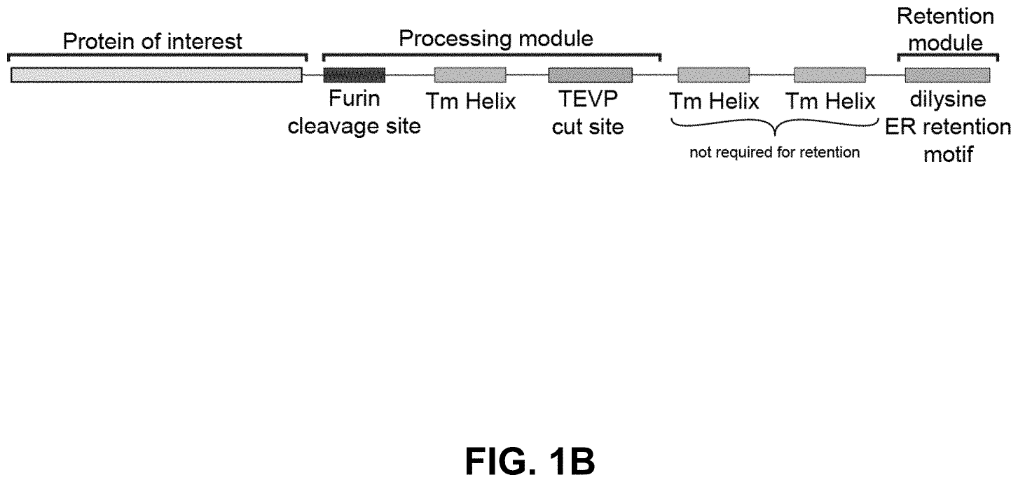

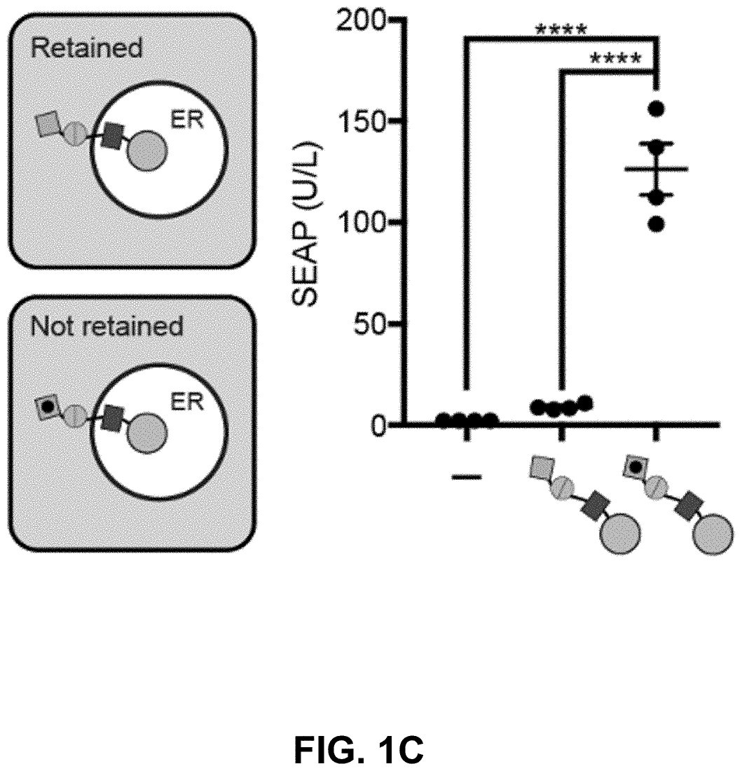

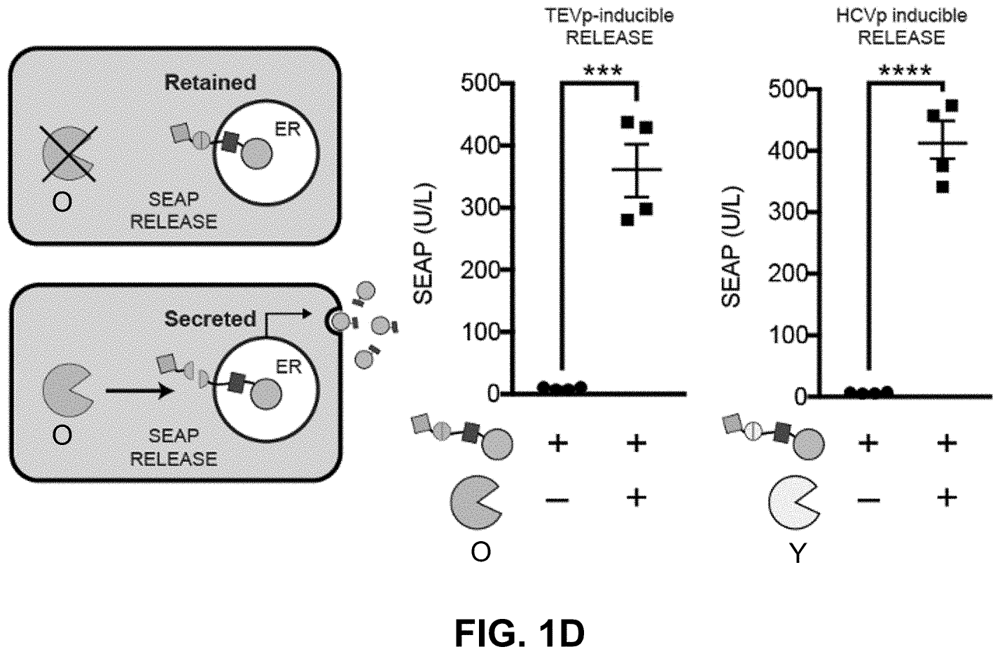

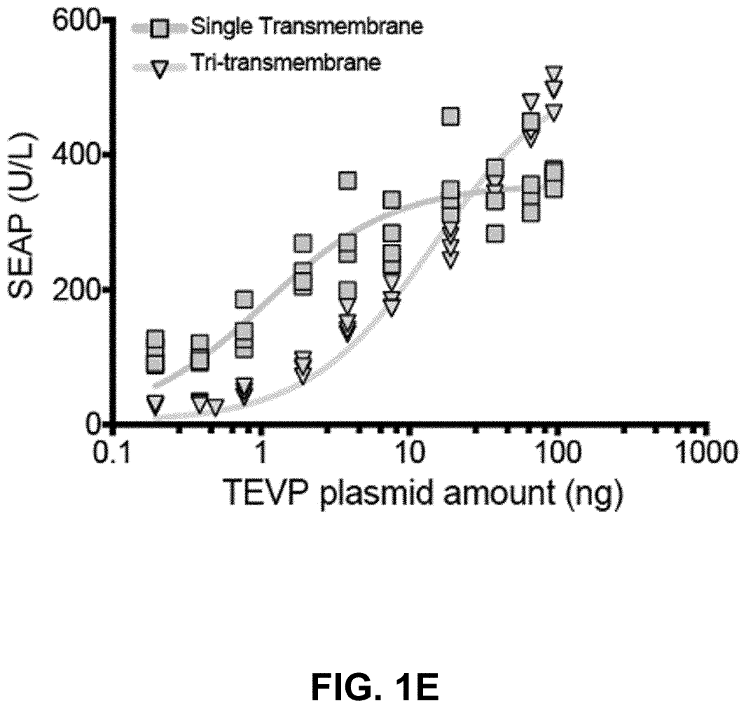

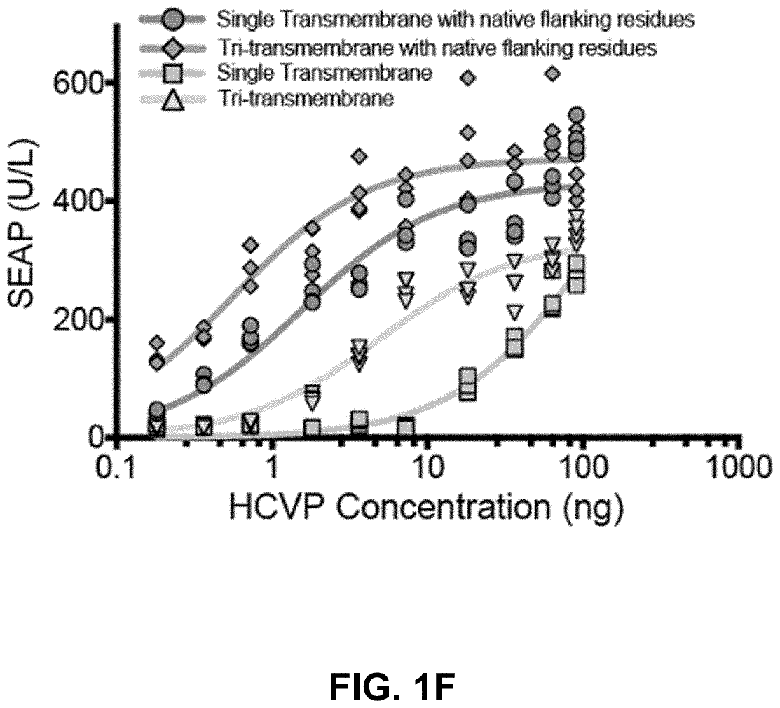

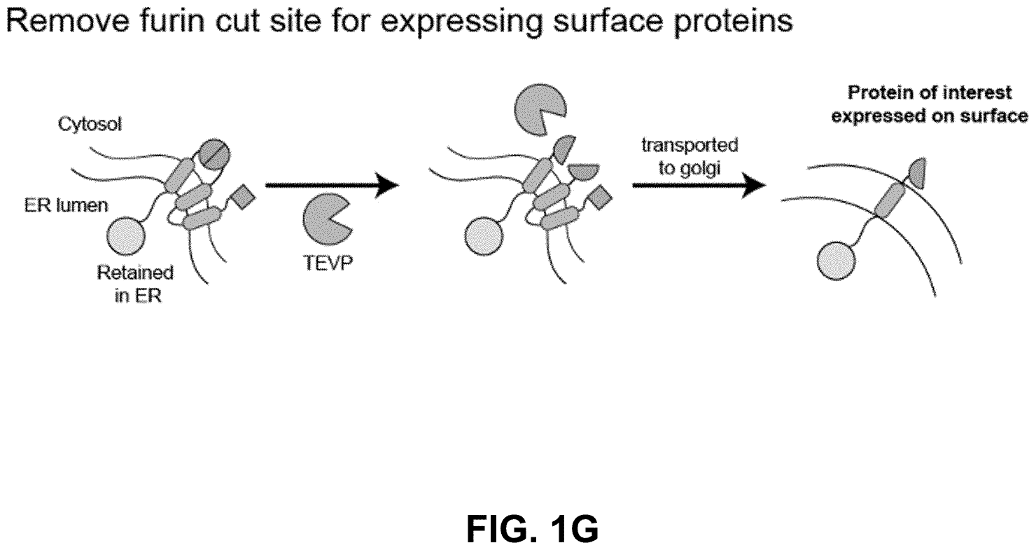

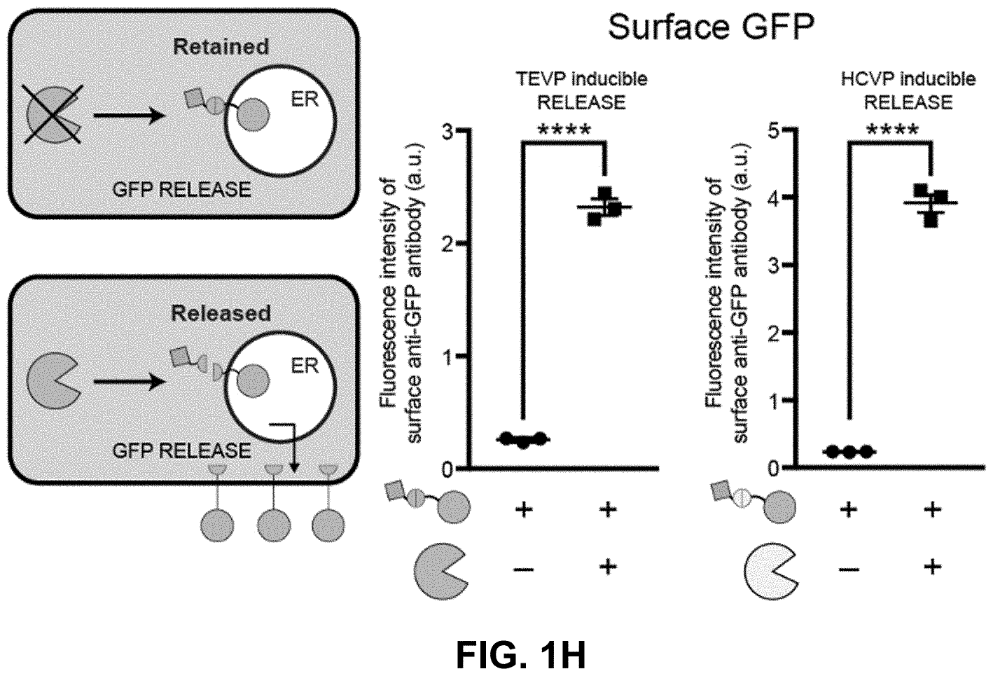

RELEASE contains 4 components (FIGS. 1A-B):
a luminal facing linker containing a furin endoprotease cut site,
a transmembrane anchor domain,
a cytosolic linker containing a protease cleavage site, and
an ER retention motif.

On the cytosolic face, the retention motif ensures that the tagged protein is actively transported back to ER, a process only aborted after the motif is removed by a proteases such as tobacco etch virus protease (TEVP).

On the luminal face, soluble proteins are initially tethered to the membrane through the linker and thus coupled to the cytosolic ER retention signal. After the first cytosolic cleavage event, the membrane-tethered protein is processed into its soluble form through cleavage by furin in the trans-Golgi apparatus (furin is absent in cis-Golgi or ER) (FIG. 1A), and finally secreted.

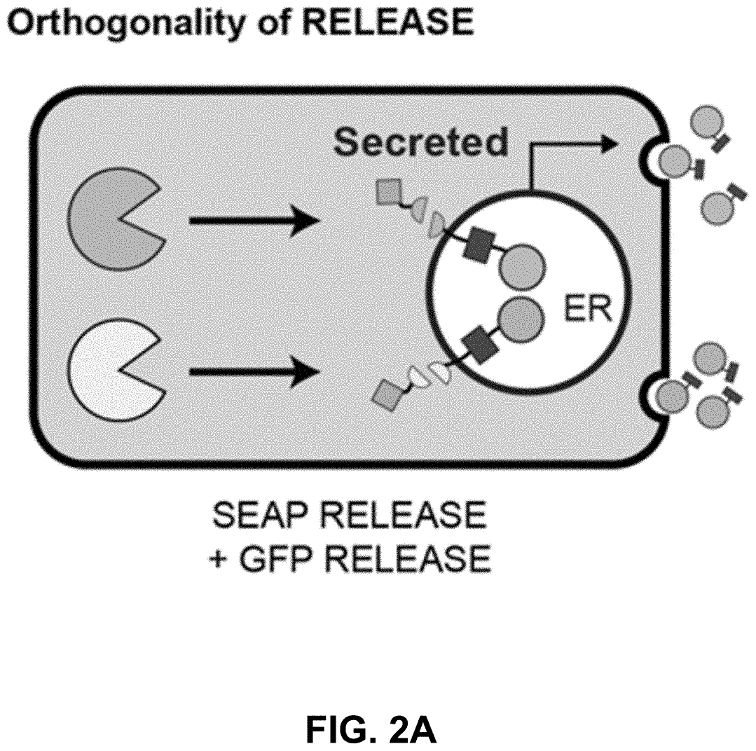

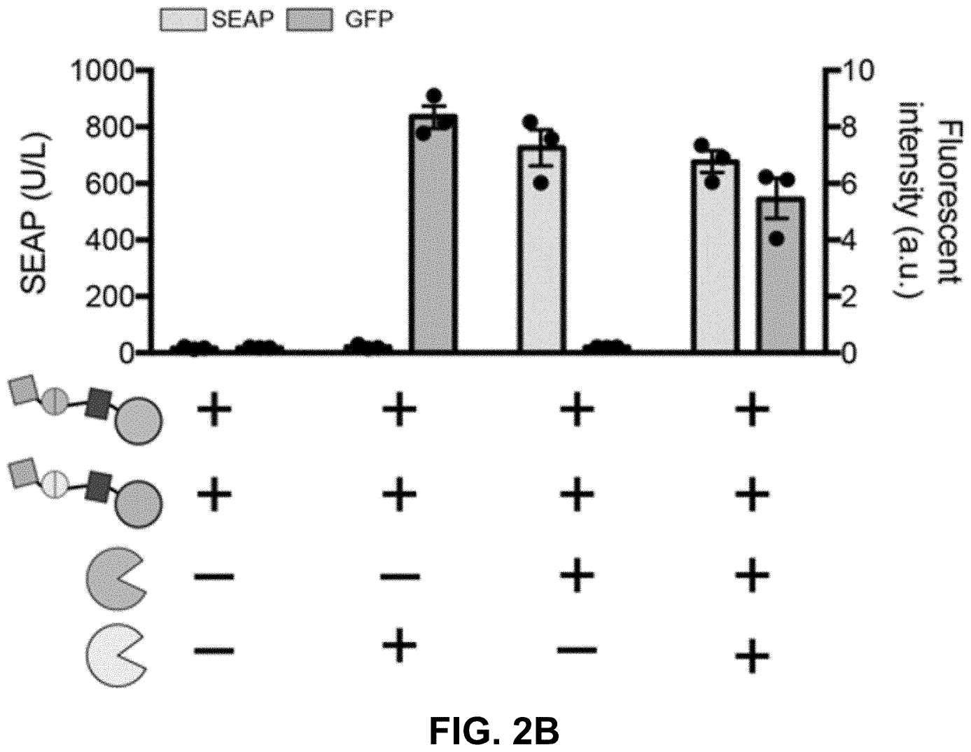

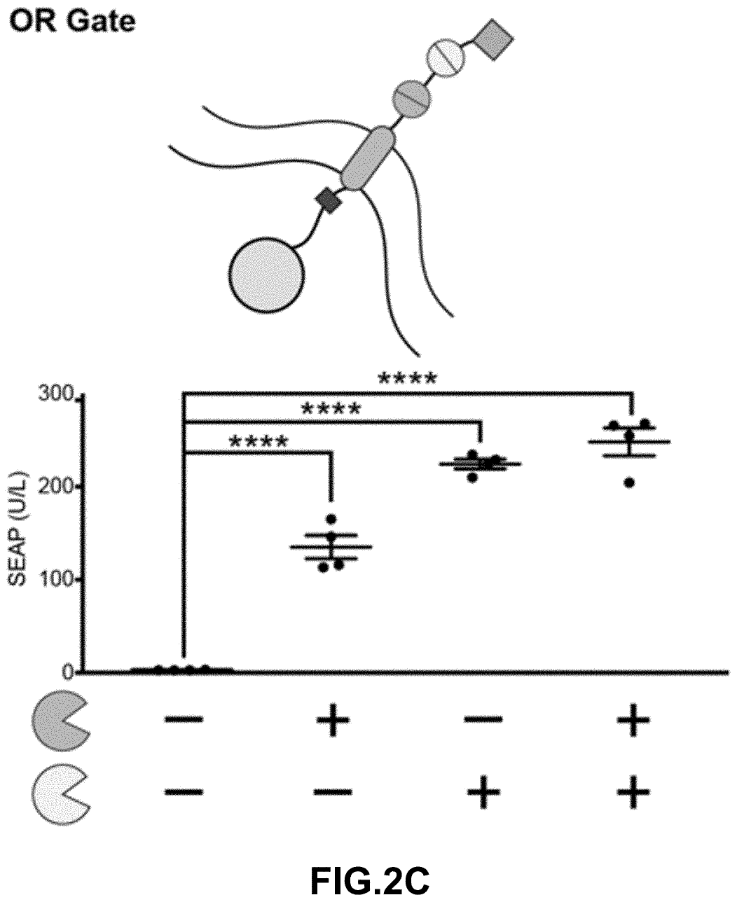

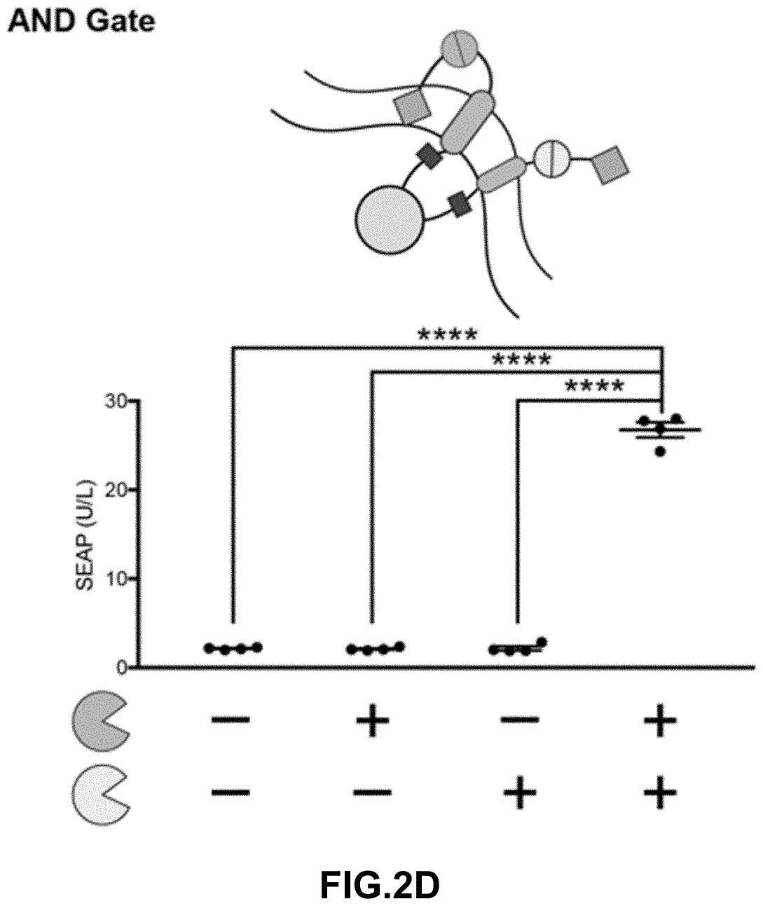

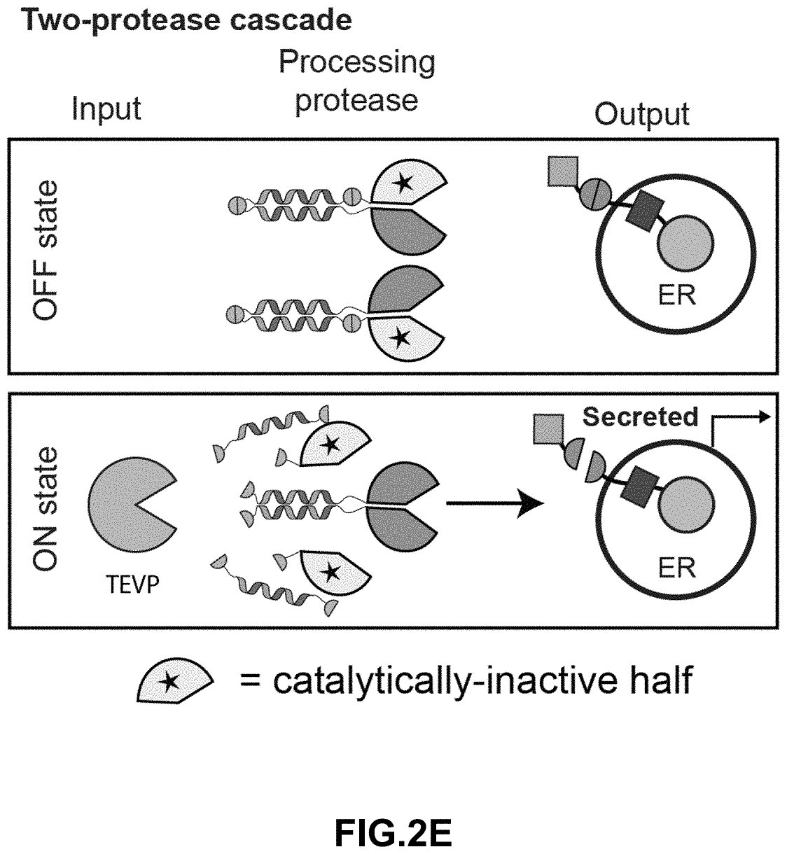

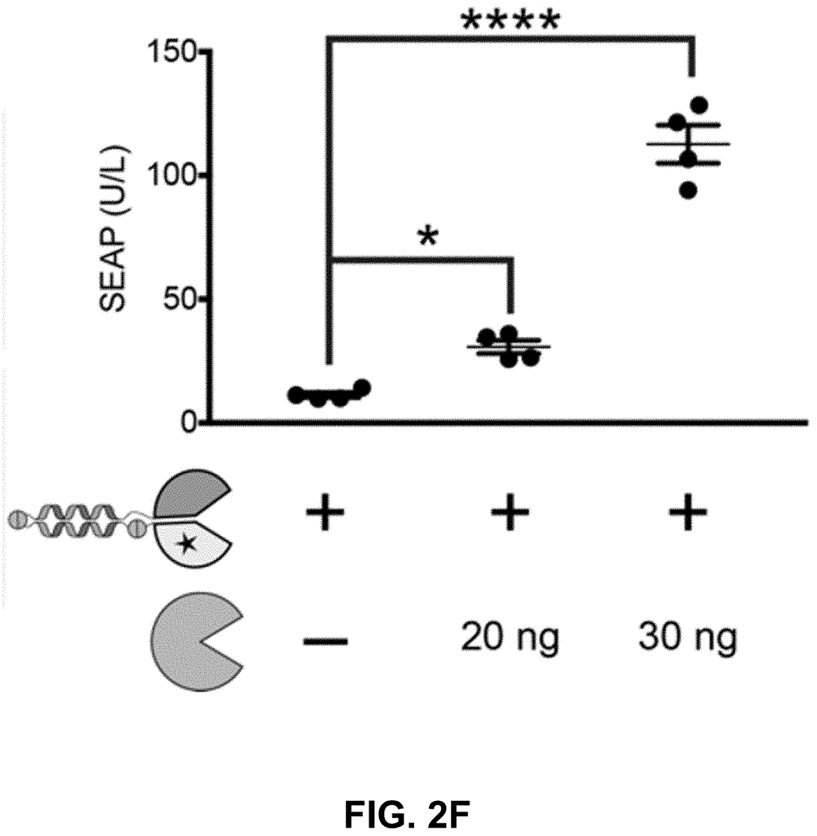

First, to validate the effectiveness of the retention motif, the inventors fused it to Secreted Embryonic Alkaline Phosphatase (SEAP), and used a dilysine-lacking mutant motif as the negative control. Human embryonic kidney (HEK) 293 cells were transfected using DNA plasmids encoding the constructs. Using RELEASE, SEAP is minimally present in the supernatant and comparable to control cells that were not transfected with SEAP (FIG. 1C). Mutation of the dilysine motif of RELEASE significantly increases SEAP secretion (FIG. 1C). Next the dilysine motif was placed under the control of TEVP, and showed that the co-expression of TEVP significantly increases SEAP secretion (FIG. 1D—left panel). By switching the cytosolic protease cut sites, RELEASE was validated against other orthogonal proteases such as the hepatitis C virus protease (HCVP) (FIG. 1D—right panel) and the tobacco mottling vein virus protease (TVMVP) (see supplementary FIGS. 1a, 1b in U.S. Provisional Patent Application 63/282,689 filed Nov. 24, 2021, which is included by reference). Furthermore, the design is compatible with alternative ER-retention motifs, as the inventors validated constructs using the N-terminal signal anchor sequence from p450 (see supplementary FIGS. 2a, 2b in U.S. Provisional Patent Application 63/282,689 filed Nov. 24, 2021, which is included by reference).

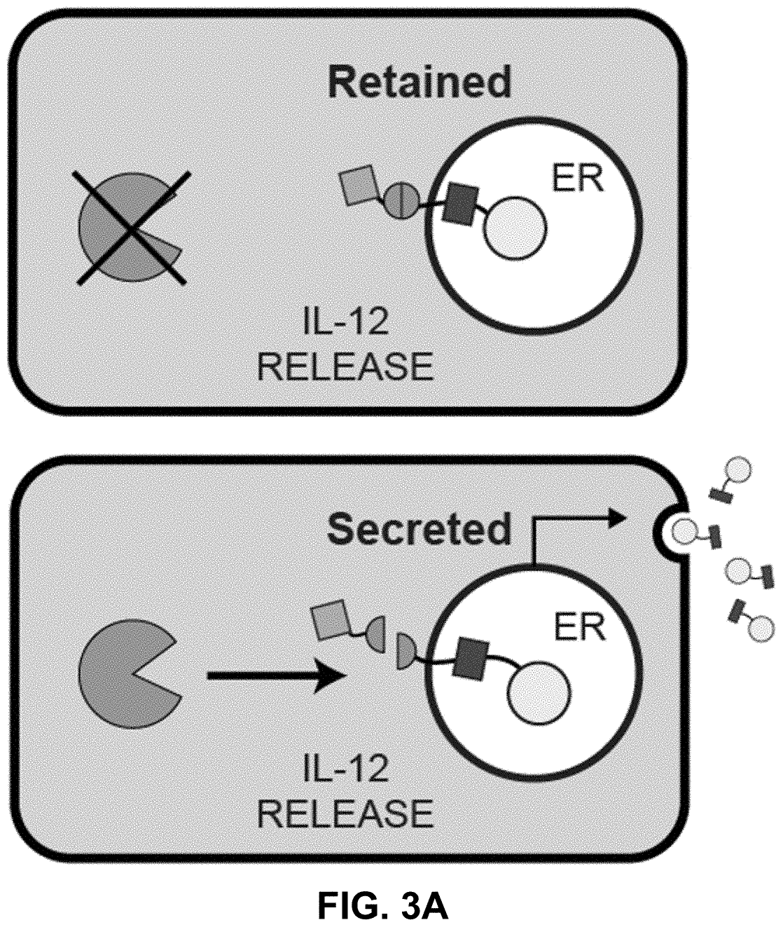

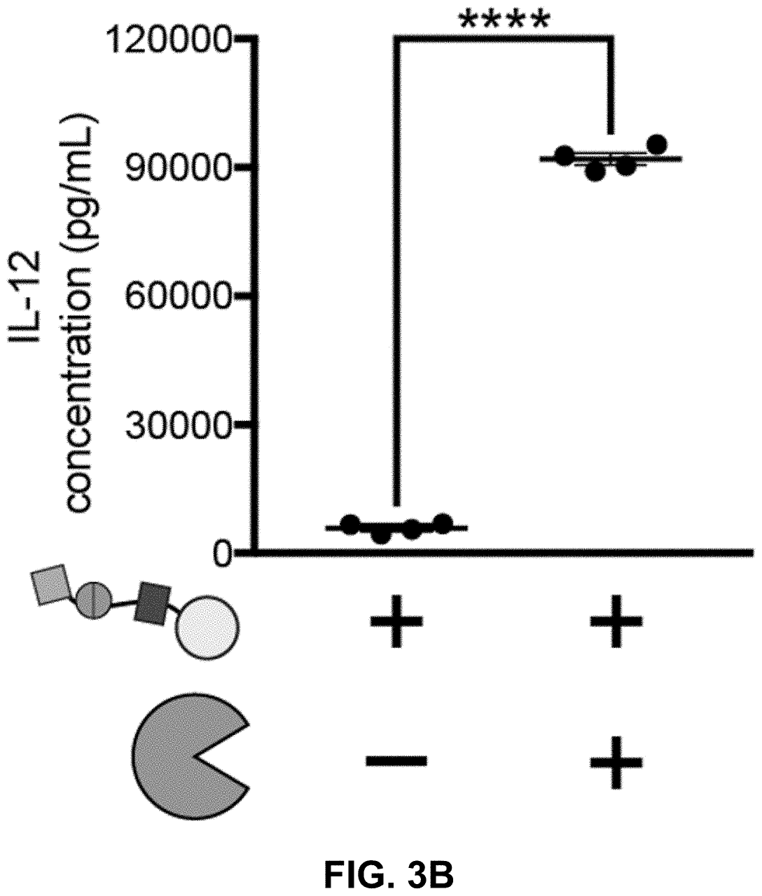

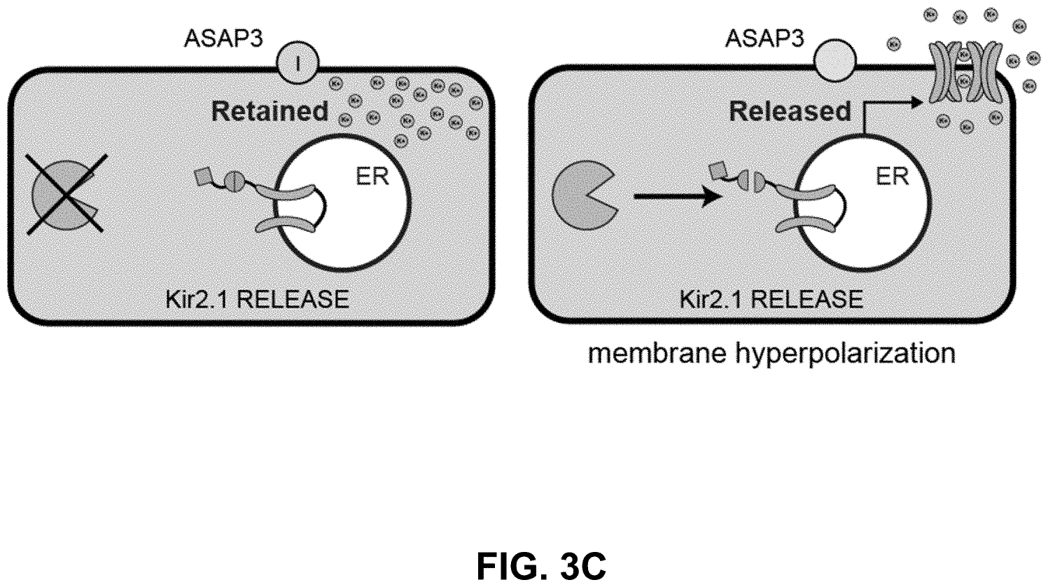

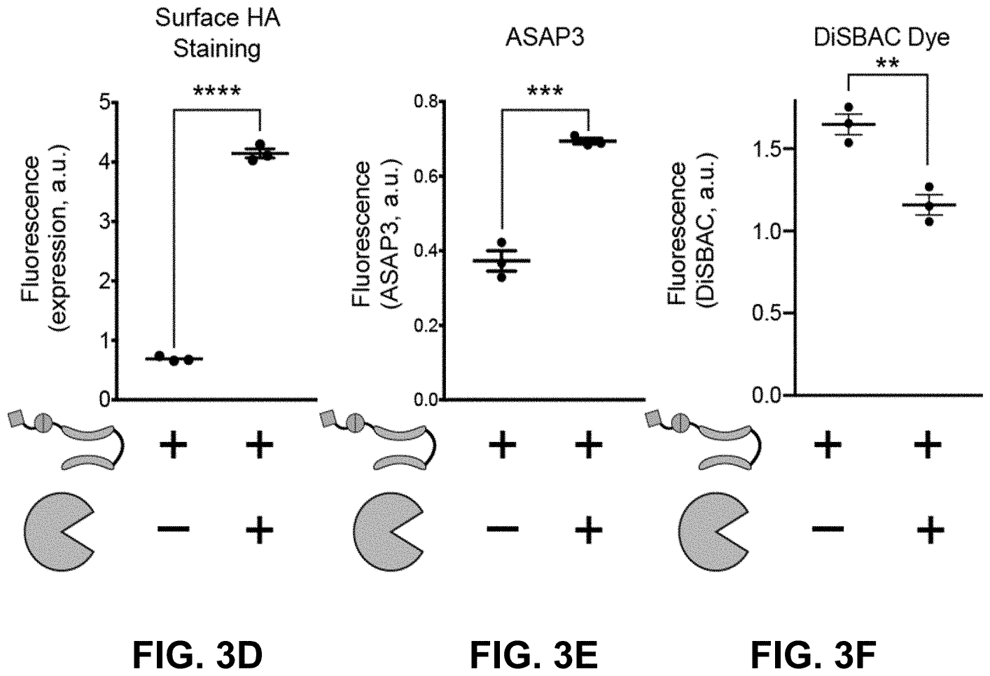

In anticipation of tuning RELEASE for different applications, the inventors next explored how its performance is affected by two design decisions. First, as an alternative to the tri-transmembrane domain, a single transmembrane variant was created, and found it more sensitive to TEVP compared to the tri-transmembrane construct (FIG. 1E). Similarly, the input sensitivity of HCVP-inducible RELEASE is also modulated by the choice of the transmembrane domain (FIG. 1F). Furthermore, by using a protein linker containing the native residues that flank the HCVP cut site, the inventors made more sensitive HCVP-inducible RELEASE constructs (FIG. 1F—diamond and circle lines) than the original versions that use synthetic flanking sequences. A complete list of the cleavage efficiencies for the RELEASE variants are shown in U.S. Provisional Patent Application 63/282,689 filed Nov. 24, 2021, Supplementary Table 1, which is included by reference. The inventors took advantage of this tunability to reduce RELEASE response to the input-independent activity of a membrane-localized split protease (see supplementary FIG. 3a in U.S. Provisional Patent Application 63/282,689 filed Nov. 24, 2021, which is included by reference) and therefore improve output dynamic range (see supplementary FIGS. 3b, 3c in U.S. Provisional Patent Application 63/282,689 filed Nov. 24, 2021, which is included by reference).

In addition to controlling protein secretion, cells can communicate by changing the display of proteins on their surface. By removing the furin cut site in RELEASE, it was hypothesized that it could control the surface display of proteins (FIG. 1G). To validate this strategy, membrane-bound green fluorescent protein (GFP) fused to RELEASE was transfected into HEK293 cells, and the cell surface was stained using an anti-GFP antibody. GFP-RELEASE constructs significantly increased surface display of GFP in response to the cognate proteases (FIG. 111). Taken together, these results show that RELEASE is a suitable approach to control the secretion and surface display of proteins in response to protease activity (FIGS. 1D, 1H).

RELEASE is Compatible with Circuit-Level Functions

After validating the RELEASE design, the next goal was to ensure that its activation could be programmed using protease-based circuits, either pre-existing or novel. For RELEASE to operate properly in circuits with multiple proteases, first it is important to validate the orthogonal control of RELEASE by the selected protease. Indeed, cells simultaneously transfected with two RELEASE constructs (FIG. 2A) were orthogonal and only secreted the respective reporter protein in response to the cognate protease (FIG. 2B). This result demonstrates that two proteases can be used to independently regulate secretion of distinct target proteins in the same cell.

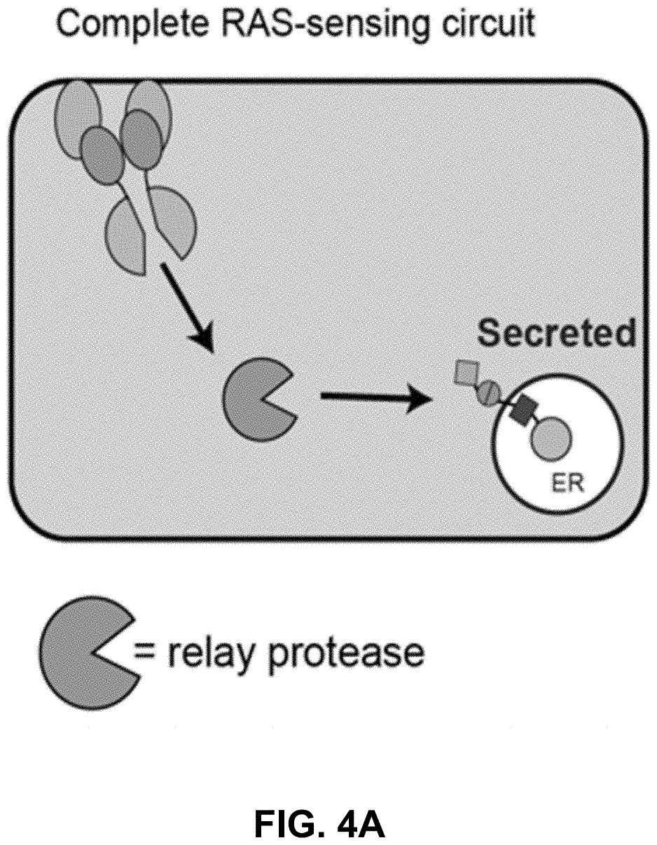

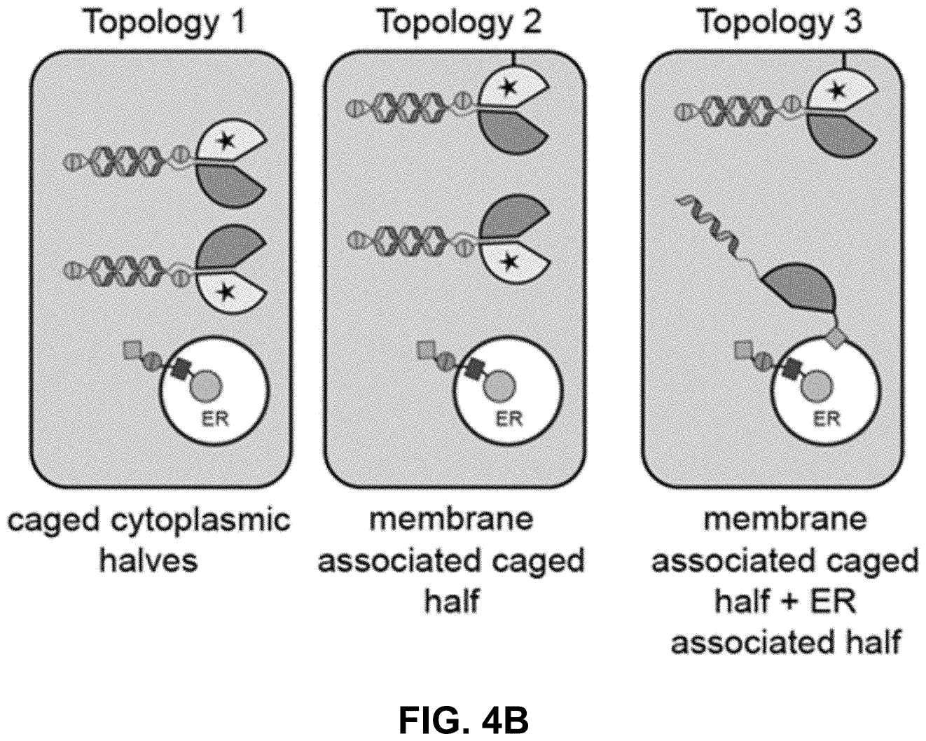

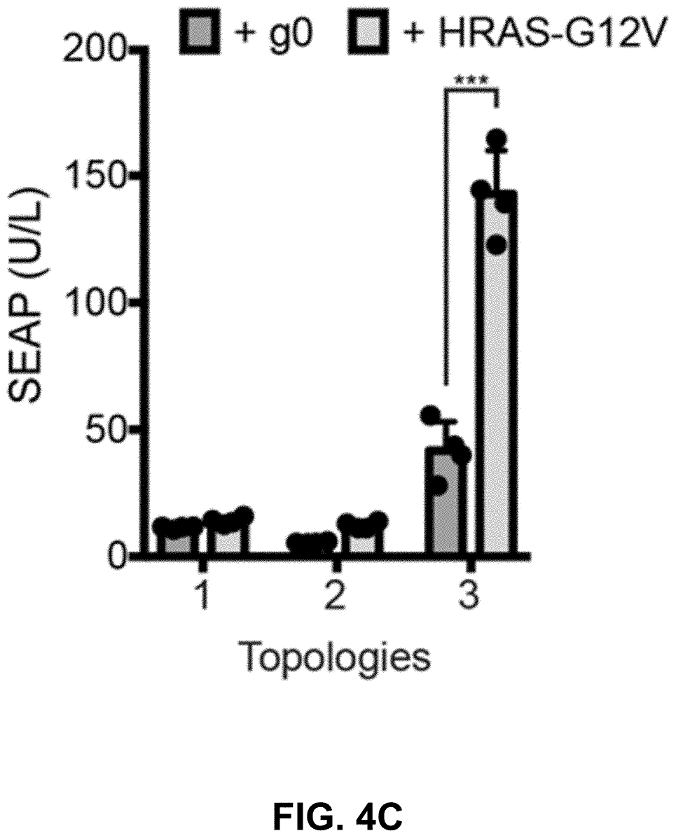

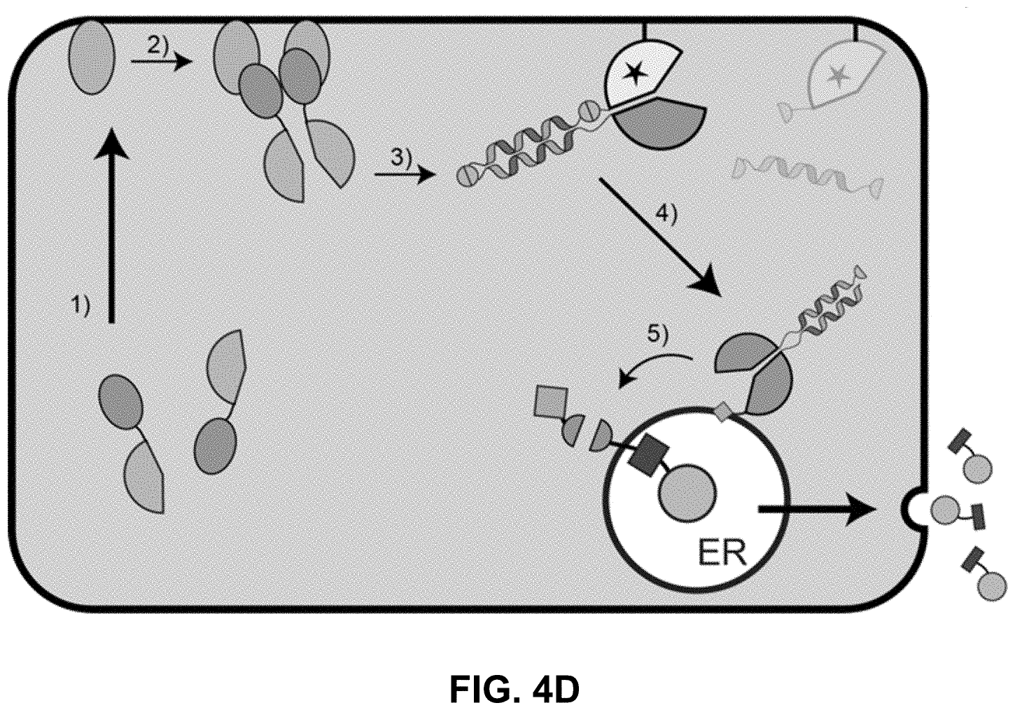

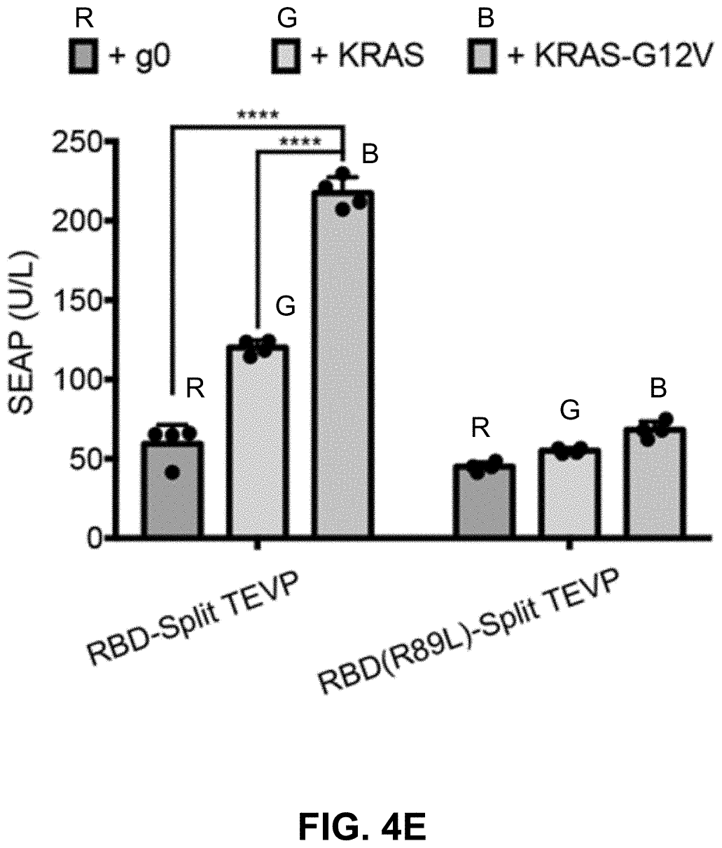

In addition to the parallel regulation of multiple outputs, another useful capability is the integration of multiple inputs. Logic operation is crucial for integrating multiple signals, previously implemented for protease circuits using degrons or coiled-coiled (CC) dimerization domains. RELEASE enables the compact implementation of Boolean logic directly at the retention level. To implement OR, two protease cut sites were inserted in tandem into the cytosolic linker so that the retention motif is removed by either protease (FIG. 2C). To implement AND, a RELEASE complex was created containing the N-terminal p450 signal anchor sequence and the C-terminal dilysine motif, each alone conferring sufficient ER retention (FIG. 2D). For SEAP to be secreted, both motifs must be removed (FIG. 2D). The inventors attributed the reduced secretion in the AND gate construct due to the use of the N-terminal signal anchor sequence (see supplementary FIG. 2b in U.S. Provisional Patent Application 63/282,689 filed Nov. 24, 2021, which is included by reference), which confers retention by directly inserting into the ER membrane rather than retention through retrograde transport. Both gates function as expected (FIGS. 2C-2D). An alternative approach for AND was also implemented (see supplementary FIG. 4 in U.S. Provisional Patent Application 63/282,689 filed Nov. 24, 2021, which is included by reference).

The inventors raised the question if other than processing signals on its own, whether RELEASE could be coupled to other protease circuits. Protease-activated protease was used as an example of such circuits. Paired CC domains were used to associate split protease halves with complementary catalytically-inactive halves (FIG. 2E), “caging’ them by preventing the active halves from associating with each other. Cut sites were incorporated adjacent to (or within) the linker regions, allowing the input protease to remove the inhibitory domains. Following removal of the autoinhibitory portion, the complementary CC domains of the functional split protease halves would then associate and reconstitute protease activity (FIG. 2E). Using this approach, a two-protease cascade was created, in which TEVP activates TVMVP, which in turn cleaves the TVMVP-inducible RELEASE. This circuit increased SEAP secretion in response to TEVP, while maintaining strong retention in the absence of TEVP (FIG. 2F). This highlights the modularity of the RELEASE design and the ability to engineer additional functionality into it.

RELEASE Controls Biologically Relevant Proteins

Many cytokines are pleiotropic and their systemic administration would cause serious adverse effects, so controlling their local expression with RELEASE would be advantageous for tumor immunotherapy. Interleukin 12 p70 (referred to as IL-12) was selected, because it is a immunomodulatory cytokine important for T-cell activation and proliferation. IL-12 is composed of two obligatory subunits (p35 and p40), so the inventors fused the two subunits with a flexible linker and then with RELEASE (FIG. 3A). As expected, TVMVP significantly increases IL-12 secretion (FIG. 3B).

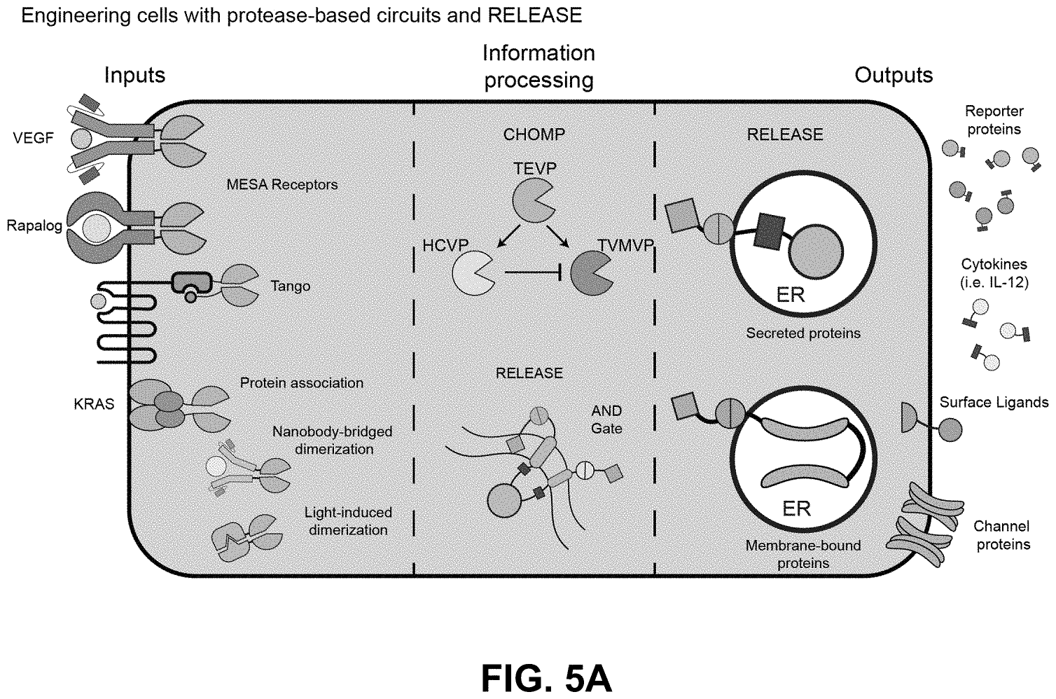

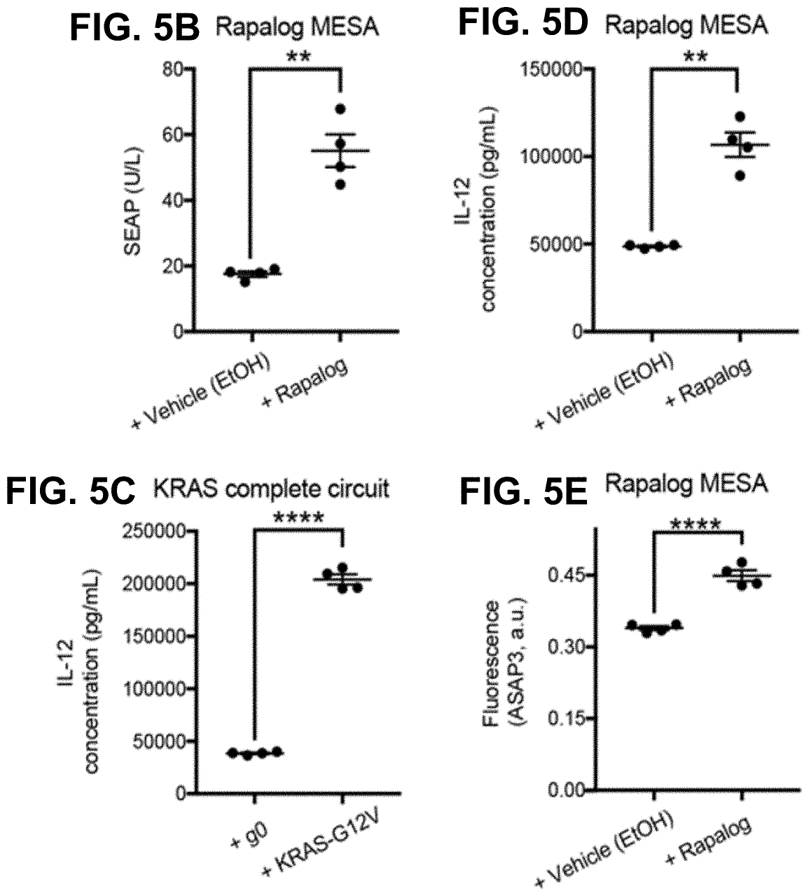

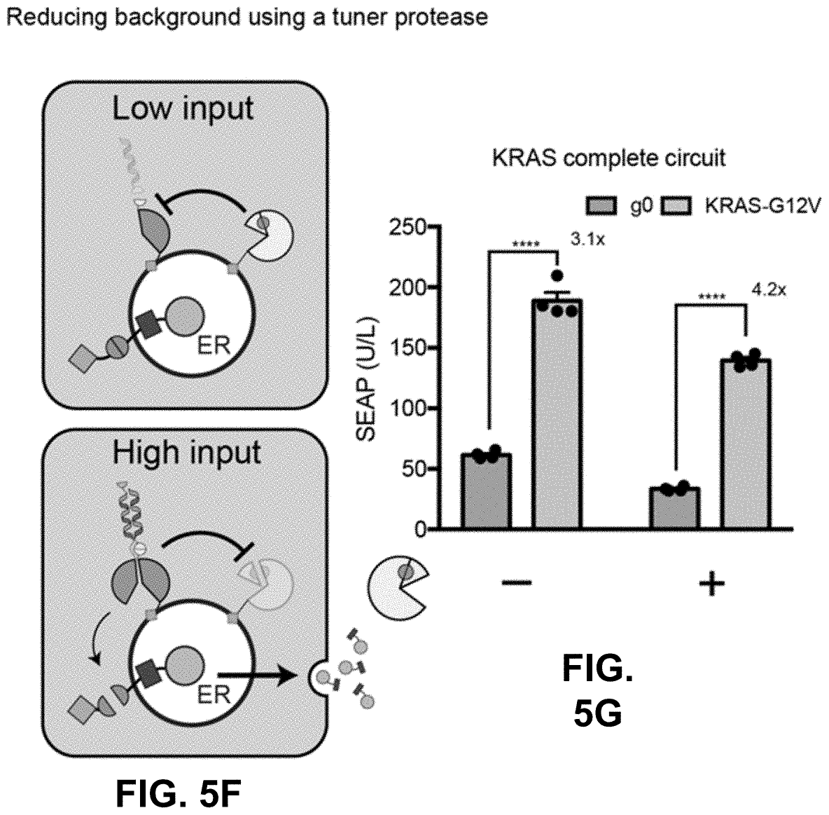

As for controlling membrane proteins, the Kir2.1 potassium channel was chose as an example of (FIG. 3C), because it is a powerful tool in neurobiology and a well-characterized model membrane protein. A protease-controlled Kir2.1 would enable the conditional silencing of neurons based on their intracellular states or extracellular cues, e.g., therapeutic silencing of the most active neurons during a seizure without the side effects of conventional methods that exert indiscriminate silencing. Unlike secreted proteins, Kir2.1 has cytosolic motifs that directs its transport in the secretory pathway, posing unique challenges for RELEASE and serving as a test case for its future adaptation to other membrane proteins. To measure the surface display of Kir2.1, a hemagglutinin (HA) epitope was incorporated into its extracellular loop. Initial experiments fusing Kir2.1 with the standard RELEASE construct resulted in leaky display of Kir2.1 in the absence of TEVP (see supplementary FIG. 5 in U.S. Provisional Patent Application 63/282,689 filed Nov. 24, 2021, which is included by reference). The inventors reasoned that it is because Kir2.1 has a long cytosolic tail, and that the dilysine motif is the most effective when positioned closely to the ER membrane. In contrast, another ER retention motif, RXR, is most effective when positioned distally from the membrane. Indeed, a RELEASE construct using the RXR motif, improved retention (see supplementary FIG. 5 in U.S. Provisional patent application 63/282,689 filed Nov. 24, 2021, which is included by reference), and successfully controlled its surface display using TEVP (FIG. 3D).

Kir2.1 functions as a homo-tetramer, provoking the question of whether the RELEASE system could interfere with tetramerization and consequently channel function (FIG. 3C). Surface display of functional Kir2.1 leads to efflux of potassium ions and hyperpolarization, providing a metric one could use to assess the its functionality. Two reporters were used to measure changes in membrane potential: ASAP3 and DiSBAC₂(3). ASAP3 is a genetically encoded voltage indicator that increases fluorescence as cells become hyperpolarized, while DiSBAC₂(3) is a chemical dye that decrease cell entry and therefore fluorescence intensity upon hyperpolarization. When Kir2.1 RELEASE was co-expressed with ASAP3, a significant increase was observed in fluorescence intensity in response to TEVP (FIG. 3E), suggesting Kir2.1 was functional. The chemical dye DiSBAC₂(3) showed similar results (FIG. 3F), and the observed change in median fluorescent intensity was indicative of a 30 mV change in membrane potential. Thus RELEASE-regulated Kir2.1 maintains its functionality.

RELEASE Responds to Oncogenic Inputs

One of the most compelling cases for protein circuits is therapy against recalcitrant cancers. The RAS family of proteins (HRAS, KRAS, and NRAS) provide a remarkable example. The activating RAS mutations have been implicated in a multitude of hard-to-treat cancers such as pancreatic ductal adenocarcinoma and non-small lung cancer. The pharmacological targeting of RAS has been challenging. The inventors envisioned a “circuit as medicine” alternative, where an intracellularly introduced circuit interrogates the cancerous state of a cell, and conditionally lyses RAS-mutant cells, while programming cytokine secretion to activate a broader local immune response.

As a first step towards that vision, the inventors hypothesized that we could exploit protein interaction during RAS signaling to activate RELEASE. RAS resides in the cell membrane, and activated RAS recruits to the membrane effector proteins such as Raf. To sense active RAS, the N- and C-terminal halves of split TEVP were fused to the RAS-binding domain (RBD) of Raf (FIG. 4A). The increased local concentration of the RBD-split TEVP sensor in response to activated RAS, along with their transition from the 3D cytosol to the more restrictive 2D membrane, was expected to facilitate the association of the protease halves through their residual mutual affinity.

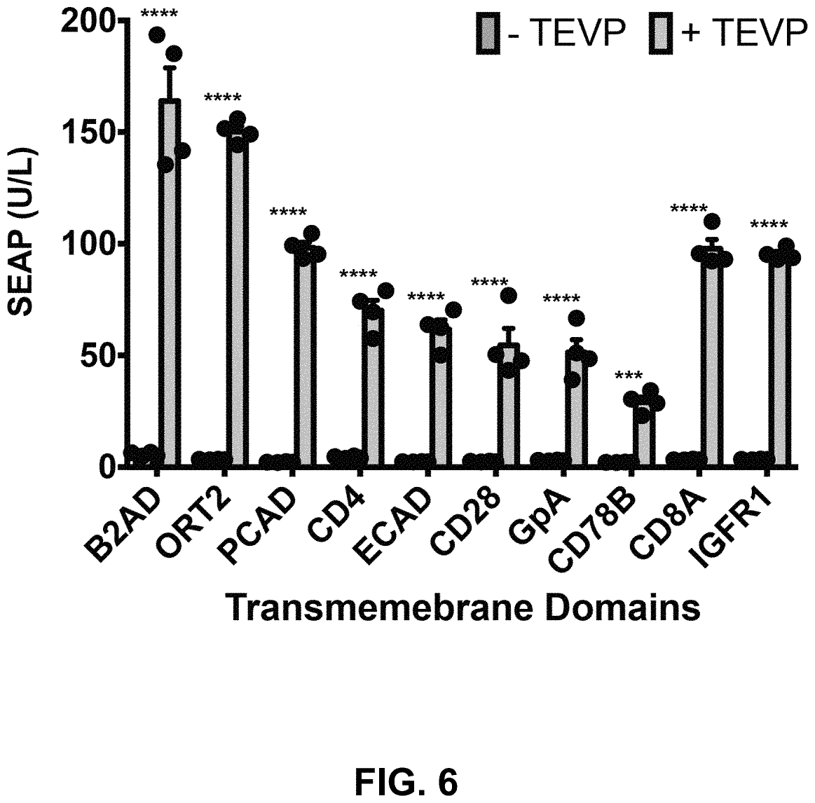

Building on inventors' previous constructs sensing the RAS pathway, experiments were performed using HRAS-G12V and the RBD-split TEVP sensor, and a minimal increase of SEAP secretion was observed when regulated by TEVP-responsive RELEASE (see supplementary FIG. 6 in U.S. Provisional patent application 63/282,689 filed Nov. 24, 2021, which is included by reference). Since HRAS-G12V reconstitutes RBD-split TEVP at the cell membrane, and cleavage of RELEASE occurs at the ER, the inventors hypothesized that additional protease components would be required to propagate the signal from the cell membrane to the ER (FIG. 4A). Using the caged TVMVP intermediate protease (FIGS. 2D, 4B—topology 1) did not improve SEAP secretion in response to HRAS-G12V, so the inventors further hypothesized that spatial localization of the intermediate protease might be required to increase signal transduction. The inventors first tried to increase the cleavage of the intermediate protease by bringing it closer to the TEVP input, fusing the C-terminal membrane transfer CAAX motif (see supplementary FIG. 7a—left panel in U.S. Provisional Patent Application 63/282,689 filed Nov. 24, 2021, which is included by reference) to one half of the caged split TVMVP (FIG. 4B—topology 2), but this did not improve SEAP secretion (FIG. 4C). The inventors then also increased the possibility for the reconstituted intermediate protease to activate RELEASE, by fusing the uncaged other half of TVMVP with the signal anchor sequence of cytochrome p450 and therefore targeting it to the ER membrane (FIG. 4B—topology 3). This resulted in the greatest SEAP secretion in response to HRAS-G12V (FIG. 4C). After titrating down the ER-bound uncaged half of TVMVP, the inventors reduced background and improved dynamic range (see supplementary FIG. 7b in U.S. Provisional Patent Application 63/282,689 filed Nov. 24, 2021, which is included by reference).

The inventors then generalized the design to KRAS, the most frequently mutated RAS in cancer. They validated that the circuit responds very similarly to KRAS-G12V and HRAS-G12V (see supplementary FIG. 7c in U.S. Provisional patent application 63/282,689 filed Nov. 24, 2021, which is included by reference), probably because RAS isoforms share up to 90% homology in the region where RBD binds. As a control, the split TEVP sensor fused to the RBD mutant (R89L), which has a reduced affinity to activated RAS, did not significantly increase SEAP secretion in response to HRAS-G12V or KRAS-G12V (FIG. 4E).

The inventors reasoned that the choice of cell membrane-localization domains might affect baseline, because post-translational modification of CAAX initially inserts the protein at the ER membrane, which could facilitate TVMVP reconstitution in the absence of TEVP inputs. To further reduce the background of the RAS sensor, they additionally tested the N-terminal membrane anchoring portion of the SH4 domain of Lyn and Fyn tyrosine kinases, the cell membrane-targeting of which bypasses ER. The Lyn and Fyn motifs reduced background SEAP secretion relative to the CAAX motif (see supplementary FIG. 7d in U.S. Provisional patent application 63/282,689 filed Nov. 24, 2021, which is included by reference), and enabled increased SEAP secretion without significantly increasing the background (see supplementary FIG. 7e in U.S. Provisional Patent Application 63/282,689 filed Nov. 24, 2021, which is included by reference).

The complete circuit is summarized in FIG. 4D. It was observed that the circuit was responsive to the oncogenic state of KRAS, since cells secreted significantly more SEAP when co-expressed with active mutants of KRAS (FIG. 4E—blue bar, see supplementary FIG. 7g in U.S. Provisional Patent Application 63/282,689 filed Nov. 24, 2021, which is included by reference) compared to wildtype KRAS (FIG. 4E—green bar), and endogenous wildtype KRAS (FIG. 4E—red bar). The oncogenic state of KRAS also resulted in a much smaller and statistically insignificant increase in SEAP secretion when using the RBD-split TEVP R89L mutant (FIG. 4E).

Plug-and-Play Capabilities of RELEASE

In addition to building towards RAS detection, our RAS-centric engineering efforts also established a plug-and-play protein circuit framework. RELEASE, in conjunction with CHOMP and other protease components, enables the detection of any input that can be converted to dimerization or proteolysis. This signal can then be processed by RELEASE itself or other protease circuits to control the display or secretion of proteins (FIG. 5A).

As a proof of principle, the inventors used the well-established MESA receptor (membrane-localized split TEVP reconstituted by rapalog) as an input to activate RELEASE via the intermediate protease circuit optimized above (FIG. 4C). Switching the input components to the rapalog MESA receptor, thye increased SEAP secretion in response to rapalog (FIG. 5B). The inventors also used RELEASE to control the secretion of IL-12 in response to mutant KRAS (FIG. 5C) or rapalog (FIG. 5D), and to control the surface display of Kir2.1 by rapalog (FIG. 5E).

The processing protease circuit is also modular. Specific applications of RELEASE may require a greater dynamic range or more complex dynamic secretion patterns that can be achieved by incorporating additional orthogonal proteases. For example, to improve the dynamic range of the RAS-sensing circuit, the inventors incorporated a previously established positive feedback loop based on reciprocal inhibition between TVMVP and HCVP to tune the level TVMVP (FIG. 5F). When input was low, or not present, HCVP would inactive the “baseline” reconstitution of TVMVP by removing the complementary CC domain (FIG. 5F—top panel). However, when there was sufficient input (KRAS-G12V⁺ cells), the reconstituted TVMVP would override HCVP by removing its activating co-peptide (FIG. 5F—bottom panel). By varying the amount of HCVP transfected, we reduced the background activity and increased the dynamic range of the engineered cells containing the complete RAS circuit (FIG. 5G). These results demonstrate the possibly of tuning RELEASE with additional proteases and eventually creating more complex responses.

Materials and Methods

Plasmid Generation

All plasmids were constructed using general practices. Backbones were linearized via restriction digestion, and inserts were generated using PCR, or purchased from Twist Biosciences. MESA-rapalog receptor source plasmids were a generous gift from Joshua Leonard. The plasmid containing the voltage indicator, ASAP3 was a generous gift from Michael Lin. A complete list of plasmids used in this study can be found in supplementary table 2 listed in U.S. Provisional Patent Application 63/282,689 filed Nov. 24, 2021, which is included by reference, and all maps will be deposited on Addgene.

Tissue Culture

Flp-In™ T-REx™ Human Embryonic Kidney (HEK) 293 cells were purchased from Thermo Scientific (Catlog #R78007). Cells were cultured in a humidity-controlled incubator under standard culture conditions (37° C. with 5% CO₂) in Dulbecco's Modified Eagle Medium (DMEM), supplemented with 10% fetal bovine serum (FBS—Fisher Scientific; catalog #FB12999102), 1 mM sodium pyruvate (EMD Millipore; catalog #TMS-005-C), 1× Pen-Strep (Genesee; catalog #25-512), 2 mM L-glutamine (Genesee, catalog #25-509) and 1×MEM non-essential amino acids (Genesee; catalog #25-536). To induce expression of transiently transfected plasmids, 100 ng/mL of Doxycycline was added at the time of transfection. Rapalog AP21967 (also known as A/C heterodimerizer, purchased from Takara Biosciences; catalog #635056) is a synthetic rapamycin analog that can bind with FRB harboring the T2098L mutation, and is designed not to interfere with the native mTOR pathway. All our constructs in this study using the FRB protein contain the T2098L mutation and were induced with 100 nM of rapalog, unless otherwise stated.

Transient Transfections

HEK 293T cells were cultured in either 24-well or 96-well tissue culture-treated plates under standard culture conditions. When cells were 70-90% confluent, the cells were transiently transfected with plasmid constructs using the jetOPTIMUS DNA transfection Reagent (Polyplus transfection, catalog #117-15), as per manufacturer's instructions.

Measuring Protein Secretion

Secreted Alkaline Phosphatase (SEAP) Assay was performed as previously described by Scheller et al. (Generalized extracellular molecule sensor platform for programming cellular behavior. Nat. Chem. Biol. 14, 723-729 (2018)). Briefly, following two days after transient transfection, the supernatant was collected without disrupting the cells and heat inactivated at 70° C. for 45 minutes. Following heat inactivation, 10-40 μL of the supernatant was mixed with dH₂O for a final volume of 80 μL, and then mixed with 100 μL of 2×SEAP buffer (20 mM homoarginine (ThermoFisher catalog #H27387), 1 mM MgCl₂, and 21% (v/v) dioethanolamine (ThermoFisher, catalog #A13389)) and 20 uL of the p-nitrophenyl phosphate (PNPP, Acros Organics catalog #MFCD00066288) substrate (120 mM). Samples were measured via kinetic measurements (1 measurement/min) for a total of 30 minutes at 405 nm using a SpectraMax iD3 spectrophotometer (Molecular Devices).

Secreted GFP was measured by incubating cell-free supernatant with cells displaying the Gbp6 GFP-binding nanobody, with mCherry fused to its cytosolic tail as a co-transfection marker. Captured GFP was used to quantify changes in the amount of secreted GFP in response to protease expression.

To measure the amount of secreted IL-12, cell-free supernatant was collected and quantified using the Human IL-12p70 DuoSet ELISA (R&D Systems; catalog #DY1270), as per the manufacturer's instructions.

Flow Cytometry and Data Analysis

Two days after transient transfection, cells were harvested using FACS buffer (HBSS+2.5 mg/mL of Bovine Serum Albumin (BSA)). For experiments requiring antibody staining, surface GFP was measured by incubating cells with a 1:1000 dilution of anti-GFP Dylight 405 antibody (ThermoFischer; catalog #600-146-215) in FACS buffer for one hour at 4° C. For experiments measuring the surface display of Kir2.1, cells were incubated with 1:500 dilution of anti-hemagglutinin antibody (HA, Abcam; catalog #ab137838), followed by incubation with a donkey anti-rabbit IgG conjugated to alexa-647 (Abcam, Cat #ab150075). After staining, cells were washed twice with FACS buffer and then strained using a 40 μm cell strainer. Cells were analyzed by flow cytometry (BioRad ZE5 Cell Analyzer). The EasyFlow Matlab-based software package developed by Yaron Antebi was used to process the flow cytometry data.

For analysis, the inventors selected and compared cells with the highest expression of the co-transfection marker, which was typically mCherry. This was done to have the largest separation between basal reporter autofluorescence from cellular autofluorescence. For experiments using the Kir2.1 potassium channel, cells were either co-transfected with the voltage indicator ASAP3 or incubated with the Oxonol chemical dye, DiSBAC2(3). The N-terminus of Kir2.1 was fused with mCherry, which acted as a co-transfection marker. After gating on cells with high expression of Kir2.1, the median fluorescence intensity was used to estimate changes in membrane potential.

Statistical Analysis

Values are reported as the means from at least 3 biological replicates, which was representative from two independent biological experiments. For experiments comparing two groups, an unpaired Student's t-test was used to assess significance, following confirmation that equal variance could be assumed (F-test). If equal variance could not be assumed, then a Welch's correction was used. For experiments comparing three or more groups, a one-way ANOVA with a post hoc Tukey test was used to compare the means among the different experimental groups. Data were considered statistically significant at a p value of 0.05. Data are presented as average±SEM, unless otherwise stated. All statistical analysis was performed using Prism 7.0 (GraphPad).

1. A composition for protease-controlled secretion of intercellular signals of a protein of interest, comprising:
(a) a transmembrane anchor domain capable of being inserted to or retained by an Endoplasmic Reticulum (ER) membrane, wherein the ER membrane distinguishes an inside to the ER membrane and an outside to the ER membrane;
(b) a luminal facing linker containing a furin endoprotease cut site, wherein the luminal facing linker is capable of making a physical connection with the protein of interest, wherein the furin endoprotease cut site is linked to the transmembrane anchor domain, and wherein once the transmembrane anchor domain is inserted to or retained by the ER membrane the luminal facing linker and the furin endoprotease cut site are located at the inside of the ER membrane;
(c) a cytosolic linker containing a protease cleavage site, wherein once the transmembrane anchor domain is inserted to or retained by the ER membrane the cytosolic linker and the protease cleavage site are located at the outside of the ER membrane; and
(d) an Endoplasmic Reticulum (ER) retention motif linked to the protease cleavage site of the cytosolic linker;
wherein at the cytosolic linker, the ER retention motif ensures that the protein of interest is actively transported back to the inside of the ER membrane, unless the ER retention motif is removed by a protease,
wherein on the luminal facing linker, the protein of interest is initially tethered to the ER membrane through the luminal facing linker and thus coupled to the cytosolic linker and the ER retention motif, and
wherein the protein of interest tethered to the ER membrane is processed into a soluble form through cleavage by furin in a trans-Golgi apparatus, and secreted.

2. A composition for protease-controlled surface expression of intercellular signals of a protein of interest, comprising:
(a) a transmembrane anchor domain capable of being inserted to or retained by an Endoplasmic Reticulum (ER) membrane, wherein the ER membrane distinguishes an inside to the ER membrane and an outside to the ER membrane;
(b) a luminal facing linker, wherein the luminal facing linker is capable of making a physical connection with the protein of interest, wherein the luminal facing linker is linked to the transmembrane anchor domain, and wherein once the transmembrane anchor domain is inserted to or retained by the ER membrane the luminal facing linker is located at the inside of the ER membrane;
(c) a cytosolic linker containing a protease cleavage site, wherein once the transmembrane anchor domain is inserted to or retained by the ER membrane the cytosolic linker and the protease cleavage site are located at the outside of the ER membrane; and
(d) an Endoplasmic Reticulum (ER) retention motif linked to the protease cleavage site of the cytosolic linker;
wherein at the cytosolic linker, the ER retention motif ensures that the protein of interest is actively transported back to the inside of the ER membrane, unless the ER retention motif is removed by a protease,
wherein on the luminal facing linker, the protein of interest is initially tethered to the ER membrane and thus coupled to the cytosolic linker and the ER retention motif, and
wherein the protein of interest tethered to the ER membrane is transported through a conventional secretory pathway, and expressed on the surface of the ER membrane.

3. An immunotherapy method using protease-controlled secretion of intercellular signals of a protein of interest, comprising:
inserting or binding a protease-controlling secretion composition to an Endoplasmic Reticulum (ER) membrane so that the protease-controlling secretion composition is retained by the ER membrane, wherein the ER membrane distinguishes an inside to the ER membrane and an outside to the ER membrane, and wherein the protease-controlling secretion composition comprises:
(i) a transmembrane anchor domain, wherein the transmembrane anchor domain is the aspect of the protease-controlling secretion composition retained by the ER membrane;
(ii) a luminal facing linker containing a furin endoprotease cut site, wherein the luminal facing linker is capable of making a physical connection with the protein of interest, wherein the furin endoprotease cut site is linked to the transmembrane anchor domain, and wherein the luminal facing linker and the furin endoprotease cut site are located at the inside of the ER membrane;
(iii) a cytosolic linker containing a protease cleavage site, wherein the cytosolic linker and the protease cleavage site are located at the outside of the ER membrane; and
(iv) an Endoplasmic Reticulum (ER) retention motif linked to the protease cleavage site of the cytosolic linker,
wherein at the cytosolic linker, the ER retention motif ensures that the protein of interest is actively transported back to the inside of the ER membrane, unless the ER retention motif is removed by a protease,
wherein on the luminal facing linker, the protein of interest is initially tethered to the ER membrane through the luminal facing linker and thus coupled to the cytosolic linker and the ER retention motif, and
wherein the protein of interest tethered to the ER membrane is processed into a soluble form through cleavage by furin in a trans-Golgi apparatus, and secreted.

4. An immunotherapy method using protease-controlled surface expression of intercellular signals of a protein of interest, comprising:
inserting or binding a protease-controlling surface expression composition to an Endoplasmic Reticulum (ER) membrane so that the protease-controlling secretion composition is retained by the ER membrane, wherein the ER membrane distinguishes an inside to the ER membrane and an outside to the ER membrane, and wherein the protease-controlling secretion composition comprises:
(i) a transmembrane anchor domain, wherein the transmembrane anchor domain is the aspect of the protease-controlling secretion composition retained by the ER membrane;
(ii) a luminal facing linker, wherein the luminal facing linker is capable of making a physical connection with the protein of interest, wherein the luminal facing linker is linked to the transmembrane anchor domain, and wherein the luminal facing linker is located at the inside of the ER membrane;
(iii) a cytosolic linker containing a protease cleavage site, wherein the cytosolic linker and the protease cleavage site are located at the outside of the ER membrane; and
(iv) an Endoplasmic Reticulum (ER) retention motif linked to the protease cleavage site of the cytosolic linker;
wherein at the cytosolic linker, the ER retention motif ensures that the protein of interest is actively transported back to the inside of the ER membrane, unless the ER retention motif is removed by a protease,
wherein on the luminal facing linker, the protein of interest is initially tethered to the ER membrane and thus coupled to the cytosolic linker and the ER retention motif, and
wherein the protein of interest tethered to the ER membrane is transported through a conventional secretory pathway, and expressed on the surface of the ER membrane.
